(19) United States
(12) Patent Application Publication
(10) Pub. No.: US 2024/0216366 A1
(43) Pub. Date: Jul. 4, 2024

(54) PHARMACEUTICAL COMPOSITION COMPRISING A DIPHENYLPYRAZINE DERIVATIVE

(71) Applicant: ACTELION PHARMACEUTICALS LTD, Allschwil (CH)

(72) Inventors: Katie AMSSOMS, Beerse (BE); Eddy DE PROOST, Beerse (BE); Wenyu DONG, Beerse (BE); Paul HARTMAN KOK, Beerse (BE); Rene HOLM, Beerse (BE); Kristof KIMPE, Beerse (BE); Greet MEURS, Beerse (BE); Maxim VERSTRAETEN, Beerse (BE)

(21) Appl. No.: 18/435,817

(22) Filed: Feb. 7, 2024

Related U.S. Application Data
(63) Continuation of application No. 18/261,923, filed on Jul. 18, 2023.

(30) Foreign Application Priority Data
Jan. 29, 2021 (WO) PCT/EP2021/052208
Nov. 24, 2021 (WO) PCT/EP2021/082830

(51) Int. Cl. A61K 31/497; A61K 9/19; A61K 47/10; A61K 47/26

(52) U.S. Cl. CPC A61K 31/497; A61K 9/19; A61K 47/10; A61K 47/26

(57) ABSTRACT

The present invention relates to a pharmaceutical composition comprising the calcium; {4-[(5,6-diphenylpyrazin-2-yl)(propan-2-yl)amino]butoxy}acetate, in particular to long-acting injectables comprising the same, the use of the pharmaceutical composition for the treatment or prevention of specific diseases, and a process to produce it.

### CROSS-REFERENCE TO RELATED APPLICATIONS

This application is a continuation of U.S. application Ser. No. 18/261,923, filed Jul. 18, 2023, which is the National Stage of International Patent Application No. PCT/EP2022/052073, filed Jan. 28, 2022, which claims the benefit of European Patent Application Number PCT/EP2021/052208, filed Jan. 29, 2021, and European Patent Application Number PCT/EP2021/082830, filed Nov. 24, 2021, the entireties of which are incorporated herein for any and all purposes.

### FIELD OF THE INVENTION

The present invention relates to a pharmaceutical composition comprising calcium; {4-[(5,6-diphenylpyrazin-2-yl)(propan-2-yl)amino]butoxy}acetate of formula (I):

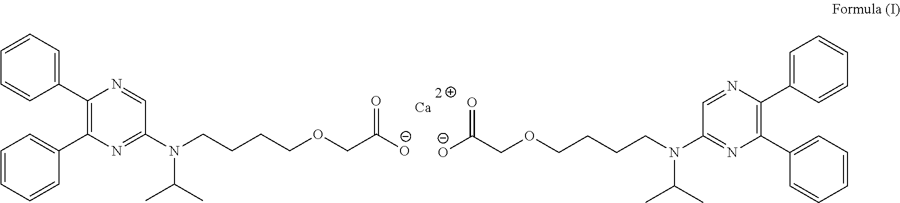

The compound of formula (I) is the calcium salt of the metabolite of selexipag (calcium salt of ACT-333679), and has the formula Ca(C₂₅H₂₈N₃O₃)₂, i.e. C₅₀H₅₆N₆O₆Ca (MW: 877.109). In the present invention, the terms “calcium; {4-[(5,6-diphenylpyrazin-2-yl)(propan-2-yl)amino]butoxy}acetate”, “calcium; 2-[4-[(5,6-diphenylpyrazin-2-yl)(propan-2-yl)amino]butoxy]acetate”, “calcium; 2-[4-[(5,6-diphenylpyrazin-2-yl)-isopropyl-amino]butoxy]acetate”, “calcium; 2-[4-[(5,6-diphenylpyrazin-2-yl)-(propan-2-yl)-amino]butoxy]acetate”, “calcium salt of {4-[(5,6-diphenylpyrazin-2-yl)(propan-2-yl)amino]butoxy}acetic acid”; “calcium salt of {4-[(5,6-diphenylpyrazin-2-yl)(isopropyl)amino]butoxy}acetic acid”; “calcium salt of 2-(4-((5,6-diphenylpyrazin-2-yl)(propan-2-yl)amino)butoxy)acetic acid”; “calcium salt of 2-(4-((5,6-diphenylpyrazin-2-yl)(isopropyl)amino)butoxy)acetic acid”; “bis[[2-[4-[(5,6-diphenylpyrazin-2-yl)-isopropyl-amino]butoxy]acetyl]oxy]calcium)” and calcium salt of the metabolite of selexipag (calcium salt of ACT-333679) are used synonymously.

Selexipag (INN) is 2-{4-[(5,6-diphenylpyrazin-2-yl)(propan-2-yl)amino]butoxy}-N-(methanesulfonyl)acetamide (ACT-293987, NS-304, CAS: 475086-01-2; 2-{4-[N-(5,6-diphenylpyrazin-2-yl)-N-isopropylamino]butyloxy}-N-(methylsulfonyl)acetamide), also known as Uptravi™. The metabolite of selexipag is 2-(4-((5,6-diphenylpyrazin-2-yl)(isopropyl)amino)butoxy)acetic acid (MRE-269, ACT-333679, 2-{4-[(5,6-diphenylpyrazin-2-yl)-propan-2-ylamino]butoxy}acetic acid; {4-[(5,6-diphenylpyrazin-2-yl)(isopropyl)amino]butoxy}acetic acid; {4-[(5,6-diphenylpyrazin-2-yl)-(propan-2-yl)amino]butoxy}acetic acid; CAS: 475085-57-5 (MW 419.52)). Salts of selexipag metabolite are described in JP 2019-149945.

The present pharmaceutical composition in the form of an aqueous suspension is suitable for intramuscular or subcutaneous injection. It may also by filled as a solid product into vials or lyophilized and reconstituted to give the respective aqueous suspension. Moreover, the present invention relates to the use of pharmaceutical compositions for the treatment or prevention of specific diseases, such as pulmonary hypertension, and, in particular, pulmonary arterial hypertension (PAH) and chronic thromboembolic pulmonary hypertension (CTEPH), and a process to produce it.

### BACKGROUND OF THE INVENTION

The preparation and the medicinal use of selexipag and its active metabolite 2-(4-((5,6-diphenylpyrazin-2-yl)(isopropyl)amino)butoxy)acetic acid is described in WO2002/088084; WO2009/157396; WO2009/107736; WO2009/154246; WO2009/157397; WO2009/157398; WO2010/150865; WO2011/024874; Nakamura et al., Bioorg Med Chem (2007), 15, 7720-7725; Kuwano et al., J Pharmacol Exp Ther (2007), 322(3), 1181-1188; Kuwano et al., J Pharmacol Exp Ther (2008), 326(3), 691-699; O. Sitbon et al., N Engl J Med (2015), 373, 2522-33; Asaki et al., Bioorg Med Chem (2007), 15, 6692-6704; Asaki et al., J. Med. Chem. (2015), 58, 7128-7137. Intravenous formulations of selexipag are disclosed in WO2018/162527. Salts of selexipag metabolites are described in JP2019-149945. US20190022004 describes liposome compositions comprising weak acid drugs and uses thereof. EP3718537 describes stealth liposomes having a prostaglandin I2 receptor agonist encapsulated therein.

Selexipag was shown to be beneficial in the treatment of pulmonary arterial hypertension. In a phase III clinical trial, among patients with pulmonary arterial hypertension, the risk of the primary composite end point of death or a complication related to pulmonary arterial hypertension was significantly lower among patients who received selexipag than among those who received placebo. Selexipag received market approval e.g. in the US and is indicated for the treatment of pulmonary arterial hypertension (PAH, WHO Group I) to delay disease progression and reduce the risk of hospitalization for PAH.

So far, standard film-coated tablet formulations of selexipag intended for twice daily oral administration have been used, wherein excipients comprise D-mannitol, corn starch, low substituted hydroxypropylcellulose, hydroxypropylcellulose, and magnesium stearate; the tablets are film coated with a coating material containing hypromellose, propylene glycol, titanium dioxide, carnauba wax along with mixtures of iron oxides.

Moreover, a safety study of the switch from oral selexipag to intravenous selexipag in patients with PAH has been conducted (NCT03187678), whereby selexipag was administered twice daily as an infusion of approximately 87 min. The dose was individualized for each patient to correspond to his/her current oral dose of selexipag.

Selexipag is thought to function as a prodrug (while retaining some agonistic activity on the IP receptor on its own) which can exert long-lasting selective IP receptor agonist activity of the active metabolite 2-(4-((5,6-diphenylpyrazin-2-yl)(isopropyl)amino)butoxy)acetic acid in mammals, especially humans. The in vivo metabolism of selexipag effectively may act as a kind of ‘slow-release mechanism’ that potentially both prolongs activity and reduces typical adverse effects associated with high concentrations of PGI2 agonists (Kuwano et al., J Pharmacol Exp Ther (2007), 322(3), 1181-1188).

In certain instances, the use of an oral formulation of selexipag may be inappropriate or impossible, e.g. in urgent care, or in case a patient is for some reasons unable to swallow a tablet.

Moreover, in general, it is desirable to reduce the drug burden, particularly for treatment regimens that may last several months or more.

The number and/or volume of dosage forms that need to be administered are commonly referred to as “drug burden”. A high drug burden is undesirable for many reasons, such as the frequency of administration, often combined with the inconvenience of having to swallow large dosage forms, as well as the need to store and transport a large number or volume of pharmaceutical formulations. A high drug burden increases the risk of patients not taking their entire dose, thereby failing to comply with the prescribed dosage regimen.

Therefore, there is a need to develop a pharmaceutical composition or formulation, whose pharmaceutical effect is maintained, for example, for one week or longer, or one month or longer, whereby it only has to be administered at long time intervals such as one week or longer, or even one month or longer (a long-acting formulation), i.e. three months.

Long-acting injectable (LAI) drug formulations that permit less frequent dosing, on the order of a week or longer, even a month or longer, are an option to address patient compliance challenges and are more convenient for the patient. Moreover, more stable drug levels in blood improve efficacy and safety. However, suboptimal physicochemical properties of the drugs often limit their formulation as conventional drug suspensions, causing problems such as stability of the suspension, as well as insufficient maintenance of therapeutically effective plasma concentrations.

Neither a long-acting formulation of selexipag or its metabolite nor a long-acting formulation for treating PAH or CTEPH has been known.

### SUMMARY OF THE INVENTION

It is an object of the present invention to provide a long-acting formulation of selexipag metabolite, 2-(4-((5,6-diphenylpyrazin-2-yl)(isopropyl)amino)butoxy)acetic acid.

The inventors of the investigational drug product described herein were tasked with creating an investigational drug product that was safe for testing in humans to eventually assess whether the investigational drug product was safe and effective to treat diseases modulated by the IP receptor, notably pulmonary hypertension and in particular PAH or CTEPH.

For example, considering the long term treatment of PAH or CTEPH, the inventors were not only charged with determining a specific and stable formulation of the investigational drug product including 2-(4-((5,6-diphenylpyrazin-2-yl)(isopropyl)amino)butoxy)acetic acid but they were also tasked with ensuring that this formulation would release 2-(4-((5,6-diphenylpyrazin-2-yl)(isopropyl)amino)butoxy)acetic acid to the PAH or CTEPH patient in need thereof for a period of at least 14 days, without exhibiting a significant burst release in the first hours/days after administration and at the same time providing over the whole release period a therapeutically effective dosage of 2-(4-((5,6-diphenylpyrazin-2-yl)(isopropyl)amino)butoxy)acetic acid to the patient.

Thus they have now found that selexipag metabolite can advantageously be formulated into a long-acting formulation by using a calcium salt of selexipag metabolite or hydrate or solvate thereof, i.e. calcium; {4-[(5,6-diphenylpyrazin-2-yl)(propan-2-yl)amino]butoxy}acetate, in a micronized form either in suspension or in solid form.

Therefore, the present invention relates to a pharmaceutical composition suitable for administration by intramuscular or subcutaneous injection, comprising calcium; {4-[(5,6-diphenylpyrazin-2-yl)(propan-2-yl)amino]butoxy}acetate or hydrate or solvate thereof in the form of an aqueous suspension. In particular, such suspension is an aqueous suspension comprising microparticles of calcium; {4-[(5,6-diphenylpyrazin-2-yl)(propan-2-yl)amino]butoxy}acetate or hydrate or solvate thereof.

The long-acting profile of the formulation allows to avoid plasma peak levels and achieves minimal toxic concentration and longer therapeutic duration.

### DESCRIPTION OF THE FIGURES

FIG. 1 shows the plasma concentrations of the different studied formulations containing selexipag, selexipag metabolite (2-(4-((5,6-diphenylpyrazin-2-yl)(isopropyl)amino)-butoxy)acetic acid; ACT333679), and the calcium salt of selexipag metabolite (calcium; {4-[(5,6-diphenylpyrazin-2-yl)(propan-2-yl)amino]butoxy}acetate; Ca-salt of ACT333679) in function of time

FIG. 2 and FIG. 3 show pK rat profiles with different particles (2, 5, 8 μm (micrometer)) of calcium salt of selexipag metabolite and different surfactants/wetting agents (Polysorbate 20 and Poloxamer 338)

FIG. 4 shows resulting particle size distributions of the differently formulated drug (calcium salt of selexipag metabolite) with different particle size

FIG. 5 shows resulting particle size distributions of the differently formulated drug (calcium salt of selexipag metabolite) with different surfactants/wetting agents

FIG. 6 shows resulting particle size distribution with calcium salt of selexipag metabolite at a concentration of 200 mg/mL

FIG. 7 shows resulting particle size distributions of the differently formulated drug (calcium salt of selexipag metabolite) with different amounts of resuspending agent

FIG. 8 shows the results of zeta potential as function of pH of suspension

### DETAILED DESCRIPTION OF THE INVENTION

The present invention is concerned with a pharmaceutical composition in the form of an aqueous suspension comprising calcium; {4-[(5,6-diphenylpyrazin-2-yl)(propan-2-yl)amino]butoxy}acetate of formula (I), or a pharmaceutically acceptable hydrate or solvate thereof:

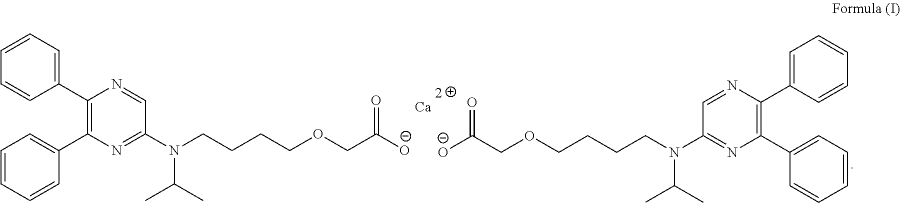

In some embodiments, the present invention is concerned with a pharmaceutical composition in the form of an aqueous suspension comprising
(a) calcium; {4-[(5,6-diphenylpyrazin-2-yl)(propan-2-yl)amino]butoxy}acetate of formula (I), or a pharmaceutically acceptable hydrate or solvate thereof:

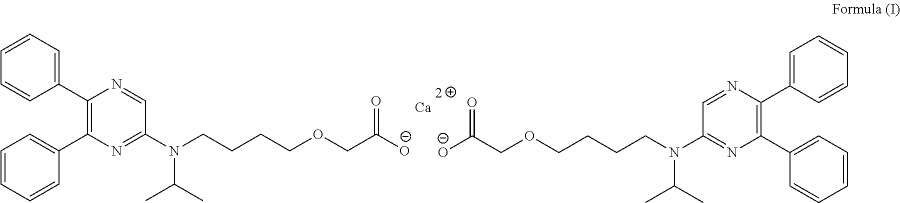

having a particle size distribution (PSD) Dv50 of 1 to 50 μm (micrometer);
(b) a surfactant and/or wetting agent;
(c) optionally, a resuspending agent; and
(d) a pharmaceutically acceptable aqueous carrier at a pH in the range of 6 to 9, and in particular in the range of 6 to 8.5.

The present pharmaceutical composition is a suspension, by which we mean that the active ingredient calcium; {4-[(5,6-diphenylpyrazin-2-yl)(propan-2-yl)amino]butoxy}acetate is suspended in the pharmaceutically acceptable aqueous carrier.

Thereby, pharmaceutical compositions in the form of an aqueous suspension are suitable for intramuscular and/or subcutaneous injection, in particular to a human patient in need thereof.

Calcium; {4-[(5,6-diphenylpyrazin-2-yl)(propan-2-yl)amino]butoxy}acetate, having the structure of formula (I) as indicated above, may be in anhydrous form, or in a hydrate form or a pharmaceutically acceptable solvate form. The term “pharmaceutically acceptable solvents” refers to solvents that retain the desired biological activity of the compound and exhibit minimal undesired toxicological effects. Preferred is an anhydrous form or a hydrate form.

Calcium; {4-[(5,6-diphenylpyrazin-2-yl)(propan-2-yl)amino]butoxy}acetate may be in a hydrate form. The hydrate form may be from about 0.1 to about 1 water molecules per calcium; {4-[(5,6-diphenylpyrazin-2-yl)(propan-2-yl)amino]butoxy}acetate molecules. In some embodiments, the molar ratio of water to calcium; {4-[(5,6-diphenylpyrazin-2-yl)(propan-2-yl)amino]butoxy}acetate ranges from about 0.1 to about 1, such as about 0.1 to about 0.15, about 0.15 to about 0.2, about 0.2 to about 0.25, about 0.25, to about 0.3, about 0.3 to about 0.35, about 0.35 to about 0.4, about 0.4 to about 0.45, about 0.45 to about 0.5, about 0.5 to about 0.55, about 0.55 to about 0.6, about 0.6 to about 0.65, about 0.65 to about 0.7, about 0.7 to about 0.75, about 0.75 to about 0.8, about 0.8 to about 0.85, about 0.85 to about 0.9, about 0.9 to about 0.95, about 0.95 to about 1. The molar ratio of water in the hydrate form may change based on storage conditions of the compound, the method of formation of the compound, and the crystal structure of the compound.

In some embodiments, calcium; {4-[(5,6-diphenylpyrazin-2-yl)(propan-2-yl)amino]butoxy}acetate or hydrate or solvate thereof is provided in a micronized form, i.e. in particles having a particle size distribution (PSD) Dv50 of 1 to 50 μm (micrometer), or 1 to 40 μm (micrometer), or 1 to 30 μm (micrometer), or 1 to 20 μm (micrometer), or 1 to 18 μm (micrometer), or 1 to 15 μm (micrometer), or 2 to 50 μm (micrometer), or 2 to 40 μm (micrometer), or 2 to 30 μm (micrometer), or 2 to 20 μm (micrometer), or 2 to 18 μm (micrometer), or 2 to 15 μm (micrometer), or 3 to 50 μm (micrometer), or 3 to 40 μm (micrometer), or 3 to 30 μm (micrometer), or 3 to 20 μm (micrometer), or 3 to 18 μm (micrometer), or 3 to 15 μm (micrometer), or 4 to 50 μm (micrometer), or 4 to 40 μm (micrometer), or 4 to 30 μm (micrometer), or 4 to 20 μm (micrometer), or 4 to 18 μm (micrometer), or 2 to 15 μm (micrometer), or 5 to 50 μm (micrometer), or 5 to 40 μm (micrometer), or 5 to 30 μm (micrometer), or 5 to 20 μm (micrometer), or 5 to 18 μm (micrometer), or 5 to 15 μm (micrometer). In some embodiments, the particle size distribution (PSD) Dv50 is 5 μm (micrometer)±10%, or 5 μm (micrometer)±5%; in some embodiments, the particle size distribution (PSD) Dv50 is 6 μm (micrometer)±10%, or 6 μm (micrometer)±5%; in some embodiments, the particle size distribution (PSD) Dv50 is 7 μm (micrometer)±10%, or 7 μm (micrometer)±5%; in some embodiments, the particle size distribution (PSD) Dv50 is 8 μm (micrometer)±10%, or 8 μm (micrometer)±5%; in some embodiments, the particle size distribution (PSD) Dv50 is 9 μm (micrometer)±10%, or 9 μm (micrometer)±5%; in some embodiments, the particle size distribution (PSD) Dv50 is 10 μm (micrometer)±10%, or 10 μm (micrometer)±5%; in some embodiments, the particle size distribution (PSD) Dv50 is 11 μm (micrometer)±10%, or 11 μm (micrometer)±5%; in some embodiments, the particle size distribution (PSD) Dv50 is 12 μm (micrometer)±10%, or 12 μm (micrometer)±5%; in some embodiments, the particle size distribution (PSD) Dv50 is 13 μm (micrometer)±10%, or 13 μm (micrometer)±5%; in some embodiments, the particle size distribution (PSD) Dv50 is 14 μm (micrometer)±10%, or 14 μm (micrometer)±5%; in some embodiments, the particle size distribution (PSD) Dv50 is 15 μm (micrometer)±10%, or 15 μm (micrometer)±5%.

The particles used herein are micro-particles, and the aqueous suspension is termed a micro-suspension, i.e. an aqueous micro-suspension.

Particle size distributions are defined herein as Dv50, also known as the median diameter. Median values are defined as the value where half of the population resides above this point, and half resides below this point. For particle size distributions the median is called the D50 (or x50 when following certain ISO guidelines). The D50 is the size in microns (micrometer, μm) that splits the distribution with half above and half below this diameter. The Dv50 (or Dv0.5) is the median for a volume distribution. The volume distribution is the primary result from laser diffraction. Herein, PSD is given in volume distribution.

Particle size distributions (PSD) can be measured by well-known methods in the art, for example, laser diffraction, sedimentation field flow fractionation, photon correlation spectroscopy or disk centrifugation.

Thereby, laser diffraction measures particle size distribution by measuring the angular variation in intensity of light scattered as a laser beam passes through a dispersed particulate sample.

Large particles scatter light at small angles relative to the laser beam and small particles scatter light at large angles. Larger particles scatter light more intense than smaller particles and will be more intensively presented in the output of the LD analysis, the volumetric size distribution. The angular scattering intensity data is then analysed to calculate the size of the particles responsible for creating the scattering pattern.

In the present application, PSD was measured with a Malvern Mastersizer 3000 apparatus from Malvern Panalytical using the laser diffraction measurement method and the Mie theory. The results of the laser diffraction analysis are reported based on the particle size volume distribution as the cumulative undersize values Dv50. The measurement method is disclosed in the experimental part.

Preferably, calcium; {4-[(5,6-diphenylpyrazin-2-yl)(propan-2-yl)amino]butoxy}acetate, having the structure of formula (I) as indicated above, is used in crystalline form.

In some embodiments, the pharmaceutical composition comprises a surfactant and/or wetting agent, or a mixture of surfactants and/or wetting agents. A “surfactant and/or wetting agent” (surfactant/wetting agent) as used herein is pharmaceutically acceptable and able to stabilise the aqueous suspension in order to avoid particle size growth during shelf-life. The surfactant and/or wetting agent may be non-ionic or ionic. Surfactants and/or wetting agents are well known in the art.

Representative examples of surfactants and/or wetting agents include gelatin, casein, lecithin, salts of negatively charged phospholipids or the acid form thereof (such as phosphatidyl glycerol, phosphatidyl inosite, phosphatidyl serine, phosphatic acid, and their salts such as alkali metal salts, e.g. their sodium salts, for example egg phosphatidyl glycerol sodium, such as the product available under the tradename Lipoid™ EPG), gum acacia, stearic acid, benzalkonium chloride, polyoxyethylene alkyl ethers, e.g., macrogol ethers such as cetomacrogol 1000, polyoxyethylene castor oil derivatives such as polyoxyl 35 castor oil (Cremophor™ EL) or polyoxyl 40 hydrogenated castor oil (Cremophor™ RH40); polyoxyethylene stearates, colloidal silicon dioxide, sodium dodecylsulfate, carboxymethylcellulose sodium, bile salts such as sodium taurocholate, sodium desoxytaurocholate, sodium desoxycholate; methylcellulose, hydroxyethylcellulose, hydroxypropylcellulose, hydroxypropylmethylcellulose, magnesium aluminate silicate, polyvinyl alcohol (PVA), poloxamers (which are block copolymers of ethylene oxide and propylene oxide), such as poloxamer 188, poloxamer 338 and poloxamer 407 (tradenames are Pluronic™ F68, F108 and F127); tyloxapol; Vitamin E-TGPS (α tocopheryl polyethylene glycol succinate, in particular α-tocopheryl polyethylene glycol 1000 succinate); poloxamines, such as Tetronic™ 908 (T908) which is a tetrafunctional block copolymer derived from sequential addition of ethylene oxide and propylene oxide to ethylenediamine; dextran; lecithin; dioctyl ester of sodium sulfosuccinic acid such as the products sold under the tradename Aerosol OT™ (AOT); sodium lauryl sulfate (Duponol™ P); alkyl aryl polyether sulfonate available under the tradename Triton™ X-200; polyoxyethylene sorbitan fatty acid esters or polysorbates (such as polysorbate 20, 40, 60 and 80, known also as Tweens™ 20, 40, 60 and 80); sorbitan esters of fatty acids (Span™ 20, 40, 60 and 80 or Arlacel™ 20, 40, 60 and 80); sucrose stearate and sucrose distearate mixtures such as the product available under the tradename Crodesta™ F110 or Crodesta™ SL-40; hexyldecyl trimethyl ammonium chloride (CTAC); polyvinylpyrrolidone (PVP), sodium dodecyl sulphate (SDS), docusate sodium, sodium deoxycholate, macrogol 15 hydroxystearate (Solutol™ HS 15), octoxynol (octoxynol-9, octoxynol-10), or simethicone. If desired, two or more surfactants and or wetting agents can be used in combination.

In one embodiment, the surfactants/wetting agents may be selected from one or more of a polysorbate, a poloxamer, an α-tocopheryl polyethylene glycol succinate, a salt of a negatively charged phospholipid (e.g. egg phosphatidylglycerols), lecithin, polyvinylpyrrolidone (PVP), docusate sodium, sodium deoxycholate, sodium dodecyl sulphate (SDS), polyoxyethylene castor oil derivatives, macrogol 15 hydroxystearate, or mixtures thereof.

Preferred surfactants/wetting agents are polysorbates, poloxamers and α-tocopheryl polyethylene glycol succinates, for example polysorbate 20, polysorbate 80, poloxamer 188, poloxamer 338, poloxamer 407, vitamin E TPGS, egg phosphatidylglycerol (Egg PG), and mixtures thereof.

Particularly preferred surfactants/wetting agents are polysorbate 20, poloxamer 338, and vitamin E TPGS, for instance polysorbate 20 and/or poloxamer 338.

Polysorbates are polyoxyethylene sorbitan fatty acid esters. Polyoxyethylene sorbitan fatty acid esters/polysorbates is the nonproprietary name, and several grades thereof are available, such as polysorbate 20, polysorbate 40, polysorbate 60, or polysorbate 80. Polysorbates are derived from ethoxylated sorbitan (a derivative of sorbitol) esterified with fatty acids. Examples for polysorbates are Polysorbate 20 (polyoxyethylene (20) sorbitan monolaurate), Polysorbate 40 (polyoxyethylene (20) sorbitan monopalmitate), Polysorbate 60 (polyoxyethylene (20) sorbitan monostearate), and Polysorbate 80 (polyoxyethylene (20) sorbitan monooleate). The different types of polysorbate differ in the fatty acid, the average number of polyoxyethylene units in the molecule and the degree of esterification. The two-digit number of the name of each polysorbate follows a certain scheme: The first number stands for the mainly esterified fatty acid: 2=lauric acid, 4=palmitic acid, 6=stearic acid, 8=oleic acid, 12=isostearic acid. The second digit indicates the type of esterification: 0 for a monoester with 20 polyoxyethylene units, 1 for a monoester with 4 or 5 polyoxyethylene units and the number 5 stands for a triester with 20 polyoxyethylene units. The preferred polysorbate 20 (CAS No 9005-64-5, E 432) is for instance sold under the brand name Tween™ 20.

Poloxamers are nonionic triblock copolymers composed of a central hydrophobic chain of polyoxypropylene (poly(propylene oxide)) flanked by two hydrophilic chains of polyoxyethylene (poly(ethylene oxide)), i.e. they are polyoxypropylene-polyoxyethylene copolymers. Preferred poloxamers are poloxamer 188, poloxamer 338, and poloxamer 407, in particular poloxamer 338.

α-Tocopheryl polyethylene glycol succinate as used herein refers to vitamin E TPGS, i.e. d-α-tocopheryl polyethylene glycol 1000 succinate, also referred to as tocophersolan (INCI), CAS No. 9002-96-4.

Lecithins are phosphatidylcholines. Herein, lecithin refers to any of a group of phospholipids, occurring in animal and plant tissues and egg yolk, composed of elements of choline, phosphoric acid, fatty acids, and glycerol.

Salts of a negatively charged phospholipid or the acid form thereof, are for example phosphatidyl glycerol, phosphatidyl inosite, phosphatidyl serine, phosphatic acid, and their salts such as alkali metal salts, e.g. their sodium salts, for example egg phosphatidyl glycerol sodium, such as the product available under the tradename Lipoid™ EPG).

Polyvinylpyrrolidone (povidone, PVP) has the molecular formula of (C₆H₉NO)ₙ. United States Pharmacopeia (USP) 32 describes povidone as a synthetic polymer consisting essentially of linear 1-vinyl-2-pyrrolidinone groups, the differing degree of polymerization of which results in polymers of various molecular weights. It is characterized by its viscosity in aqueous solution, relative to that of water, expressed as a K-value, in the range 10-120. The K-value is calculated using Fikentscher's equation. Several are available, such as PVP K12, PVP K15, PVP K17, PVP K25, PVP K30, PVP K60, PVP K90 or PVP K120. Preferred is PVPK17.

The optimal relative amount of calcium; {4-[(5,6-diphenylpyrazin-2-yl)(propan-2-yl)amino]butoxy}acetate, in relation to the surfactant/wetting agent depends on the surfactant/wetting agent selected, the specific surface area of the drug suspension which is determined by the average effective particle size and the drug concentration, the critical micelle concentration of the surfactant/wetting agent if it forms micelles, etc. The relative amount (w/w) of drug to the surfactant/wetting agent preferably is in the range 20:1 to 2:1, in particular in the range of 18:1 to 4:1.

The pharmaceutical composition optionally comprises a resuspending agent. A resuspending agent as used herein is pharmaceutically acceptable and able to stabilise the aqueous suspension in order to avoid caking during shelf-life, or needly clogging, or to facilitate resuspending the formulation after storage.

The resuspending agent is selected from the group consisting of polyethylene glycol (PEG) of various polymerization grades, carmellose sodium, and poloxamers, or a mixture thereof; preferably polyethylene glycol (PEG) of various polymerization grades, and carmellose sodium, or a mixture thereof.

Preferred resuspending agents are selected from the group consisting of PEG 4000, PEG 3350, PEG 6000, PEG 8000, PEG 20000, and carmellose sodium, or a mixture thereof; in particular PEG 4000.

It may be noted that poloxamers can function as surfactants/wetting agents, but also as resuspending agents, because they contribute some viscosity in the suspension. In one embodiment, the resuspending agent is selected from the group consisting of PEG 4000, PEG 3350, PEG 6000, PEG 8000, PEG 20000, carmellose sodium, Poloxamer 338, and

Poloxamer 407, or a mixture thereof. Preferred resuspending agents are selected from the group consisting of PEG 4000, PEG 3350, PEG 6000, PEG 8000, PEG 20000, and carmellose sodium, or a mixture thereof, in particular polyethylene glycol 4000.

Polyethylene glycol (PEG) exists in various polymerization grades. The structure of PEG is commonly expressed as H—(O—CH₂—CH₂)ₙ—OH. Polyethylene glycols (PEGs) are available in various grades, which is indicated as a number, for instance PEG 2000, PEG 3000, PEG 3350, PEG 4000, PEG 4600, PEG 6000, PEG 8000 or PEG 20000. The number is indicative for the average molecular weight of the polymer.

A preferred carmellose sodium (carboxymethylcellulose sodium) has a viscosity of 27-50 mPa·s (Viscosity 2%), 0.65 to 0.90 degree of substitution and 7.0-8.8% Na content (calculated vs DS). The product conforms to the monograph for carmellose sodium in the current European Pharmacopeia.

The optimal relative amount (w/w) of the drug, i.e. calcium; {4-[(5,6-diphenylpyrazin-2-yl)(propan-2-yl)amino]butoxy}acetate, in relation to the resuspending agent depends on the resuspending agent selected, and is preferably in the range of 2:1 to 1:3, in particular 2:1 to 1:1.

It is to be noted that each indicated surfactant/wetting agent described above may be combined with each resuspending agent mentioned herein. Particularly preferred combinations are polysorbate 20 with PEG 4000, poloxamer 338 with PEG 4000, vitamin E TPGS with PEG 4000, poloxamer 338 and carmellose sodium, polysorbate 80 and carmellose sodium, and poloxamer 338 and vitamin E TPGS.

The pharmaceutical composition comprises a pharmaceutically acceptable aqueous carrier. Said aqueous carrier comprises sterile water, i.e. water suitable for injection, optionally in admixture with other pharmaceutically acceptable ingredients. These ingredients may be selected from one or more of a buffering agent, a pH adjusting agent, a preservative or an isotonizing agent.

The aqueous carrier has a pH in the range of 6 to 8.5. Further pH ranges are from 7 to 8.5, or pH 8, i.e. pH 8±½, or pH 7.5, i.e. 7.5±½.

In one embodiment, the composition comprises one or more buffering and/or pH adjusting agent(s), rendering the pH of the aqueous carrier in the range of 6 to 8.5; 7 to 8.5, or pH 8, i.e. pH 8±½, or pH 7.5, i.e. 7.5±½.

In some embodiments, the buffering and/or pH adjusting agent(s) is/are selected from the group consisting of disodium hydrogen phosphate, citric acid, tris(hydroxymethyl)aminomethane (TRIS), histidine, HCl and NaOH, or a mixture thereof.

Thereby, the buffering agents are disodium hydrogen phosphate, citric acid, tris(hydroxymethyl)aminomethane (TRIS) and histidine; and the pH-adjusting agents are HCl or NaOH, preferably in aqueous solution. In particular, the buffering and/or pH adjusting agent(s) is/are selected from the group consisting of disodium hydrogen phosphate, citric acid, tris(hydroxymethyl)aminomethane (TRIS), HCl and NaOH, or a mixture thereof. Thereby, the buffering agents are disodium hydrogen phosphate, citric acid, and tris(hydroxymethyl)aminomethane (TRIS); and the pH-adjusting agents are HCl or NaOH, preferably in aqueous solution.

Preferably, the pharmaceutically acceptable aqueous carrier comprises citric acid. Citric acid thereby serves as buffering agent, but also as chelating agent and antioxidant.

A preferred pH of the aqueous suspension is pH 8±½. Micro-suspensions may be formulated with TRIS buffer, however, McIlvaine buffer (citric acid and disodium hydrogen phosphate) is preferred. McIlvaine buffer at pH 8±½ consists of disodium hydrogen phosphate anhydrous and citric acid, the buffer strength ranging from 5 to 100 mM. 10 to 50 mM is preferred. However, it is also possible to add more citric acid and adjust the pH with NaOH to pH 8±½.

The buffering agent or buffer is able to provide stability to the formulation, i.e. to prevent dissociation into the free from of the metabolite of selexipag, i.e. the free acetic acid derivative. The buffer strength ranges from 5 to 100 millimolar (mM), or from 10 to 50 mM.

Suitable optional preservatives for the pharmaceutical compositions comprise antimicrobials and anti-oxidants which can be selected from the group consisting of benzoic acid, benzyl alcohol, butylated hydroxyanisole (BHA), butylated hydroxytoluene (BHT), chlorbutol, a gallate, a hydroxybenzoate, EDTA, phenol, chlorocresol, metacresol, benzethonium chloride, myristyl-y-piccolinium chloride, phenylmercuric acetate and thimerosal. Radical scavengers include BHA, BHT, Vitamin E and ascorbyl palmitate, and mixtures thereof. Oxygen scavengers include sodium ascorbate, sodium sulfite, L-cysteine, acetylcysteine, methionine, thioglycerol, acetone sodium bisulfite, isoacorbic acid, hydroxypropyl cyclodextrin. Chelating agents include sodium citrate, sodium EDTA and malic acid. In one embodiment, the composition does not contain a perseverative.

An isotonizing agent or isotonifier may be present to ensure isotonicity of the pharmaceutical composition, and includes sugars such as mannitol, glucose, dextrose, sucrose, fructose, trehalose, lactose; polyhydric sugar alcohols, preferably trihydric or higher sugar alcohols, such as glycerin, erythritol, arabitol, xylitol, sorbitol and mannitol. Alternatively, sodium chloride, sodium sulfate, or other appropriate inorganic salts may be used to render the solutions isotonic. These isotonifiers can be used alone or in combination. The aqueous suspensions conveniently comprise from 0 to 10% (w/v), in particular 0 to 6% of isotonizing agent. Of interest are nonionic isotonifiers, e.g. glucose, as electrolytes may affect colloidal stability. In one embodiment, the composition contains an isotonizing agent or isotonifier, which, in a further embodiment is a nonionic isotonifier, such as a suitable sugar such as mannitol.

A desirable feature for a pharmaceutical composition relates to the ease of administration. The viscosity of the pharmaceutical composition should be sufficiently low to allow administration by injection, and sufficiently high to maintain slow sedimentation and good resuspendability. In particular it should be designed so that it can be taken up easily in a syringe (e.g. from a vial), injected through a fine needle (e.g. a 19 G to 25 G needle) in not too long a time span. In one embodiment the viscosity of the composition is from 1 mPa·s to 75 mPa·s at 200 s⁻¹, or from 5 mPa·s to 40 mPa·s at 200 s⁻¹.

Ideally, the aqueous suspension will comprise as much calcium; {4-[(5,6-diphenylpyrazin-2-yl)(propan-2-yl)amino]butoxy}acetate, or a pharmaceutically acceptable hydrate or solvate thereof, as can be tolerated so as to keep the injection volume to a minimum, in particular 2% to 50% (w/v), or from 2% to 45% (w/v), or from 2% to 40% (w/v), or from 2% to 35% (w/v), or from 2% to 30% (w/v), or from 2% to 25% (w/v), or from 2% to 20% (w/v), or from 2% to 15% (w/v), in particular from 2.5% to 10% (w/v).

Ideally, the amount of surfactant/wetting agent is selected as low as possible but effective and robust, in particular from 0.5% to 20% (w/v), or from 0.5% to 15% (w/v), or from 0.5% to 12% (w/v) or from 0.5% to 10% (w/v), or from 0.5% to 8% (w/v), or from 0.5% to 7% (w/v), or from 0.5% to 6% (w/v), or from 0.5% to 5% (w/v), or from 0.5% to 4% (w/v), or from 0.5% to 3% (w/v), or from 0.5% to 2% (w/v), of a surfactant/wetting agent, or a mixture of surfactants/wetting agents.

Ideally, the amount of resuspending agent is selected as low as possible but effective, in particular from 0% to 30% (w/v), or from 1% to 30% (w/v), or from 1% to 25%, or from 1% to 20% (w/v), or from 1 to 15% (w/v), or from 3 to 10% (w/v) of a resuspending agent or a mixture of resuspending agents.

Ideally, the amount of buffering agent is selected as low as possible but effective, in particular from 0 to 100 mM, or from 5 to 100 mM, or from 5 to 50 mM, or from 10 to 50 mM of a buffering agent, or a mixture of buffering agents.

In one embodiment, the pharmaceutical composition comprises by weight based on the total volume of composition
(a) from 2% to 50% (w/v), or from 2% to 30% (w/v), or from 2% to 15% (w/v) or from 2.5% to 10% (w/v) of calcium; {4-[(5,6-diphenylpyrazin-2-yl)(propan-2-yl)amino]butoxy}acetate (or a pharmaceutically acceptable hydrate or solvate thereof; but where the w/v is calculated on the basis of its anhydrous form);
(b) from 0.5% to 20% (w/v), or from 0.5% to 15% (w/v), or from 0.5% to 12% (w/v), or 0.5% to 10%, or from 0.5% to 8% (w/v), or from 0.5% to 7% (w/v), or from 0.5% to 6% (w/v), or from 0.5% to 5% (w/v), or from 0.5% to 4% (w/v), or from 0.5% to 3% (w/v), of a surfactant/wetting agent or a mixture of surfactants/wetting agents;
(c) from 0% to 30% (w/v), or from 1% to 30% (w/v), or from 1% to 20% (w/v), or from 1 to 15% (w/v) or from 3 to 10% (w/v) of a resuspending agent or a mixture of resuspending agents; and
(d) from 0 to 100 mM, or from 5 to 50 mM, or from 10 to 50 mM of a buffering agent, or mixtures thereof;
(e) water for injection q.s. ad 100%.

In one embodiment, the pharmaceutical composition comprises by weight based on the total volume of composition
(a) from 2% to 15% (w/v) or from 2.5% to 10% (w/v) of calcium; {4-[(5,6-diphenylpyrazin-2-yl)(propan-2-yl)amino]butoxy}acetate (or a pharmaceutically acceptable hydrate or solvate thereof; but where the w/v is calculated on the basis of its anhydrous form);
(b) from 0.5% to 12% (w/v), or 0.5% to 10%, or from 0.5% to 8% (w/v), or from 0.5% to 7% (w/v), or from 0.5% to 6% (w/v), or from 0.5% to 5% (w/v), or from 0.5% to 4% (w/v), or from 0.5% to 3% (w/v), of a surfactant/wetting agent or a mixture of surfactants/wetting agents;
(c) from 0 to 15% (w/v), or from 1 to 15% (w/v) or from 3 to 10% (w/v) of a resuspending agent or a mixture of resuspending agents; and
(d) from 5 to 50 mM, or from 10 to 50 mM of a buffering agent, or mixtures thereof;
(e) water for injection q.s. ad 100%.

Thereby, the surfactant/wetting agent, the optional resuspending agent, and buffering agents, as well as mixtures thereof, are as described above.

Calcium; {4-[(5,6-diphenylpyrazin-2-yl)(propan-2-yl)amino]butoxy}acetate can be prepared as described in the example part.

The pharmaceutical compositions as described herein may be in a container, notably in a vial or in a syringe; especially in a syringe.

In some embodiments, a pharmaceutical composition as described herein can be prepared by a process comprising the steps of:
(a) adding calcium; {4-[(5,6-diphenylpyrazin-2-yl)(propan-2-yl)amino]butoxy}acetate, or a pharmaceutically acceptable hydrate or solvate thereof, to a liquid medium comprising a surfactant and/or wetting agent at a pH in the range of 6 to 8.5, optionally a resuspending agent; and a pharmaceutically acceptable aqueous carrier, to form a premix/predispersion; and
(b) subjecting the premix to mechanical means in the presence of a grinding medium to reduce the average effective particle size.

The particle size of the micro-particles can be prepared by mechanical means known in the art. In one embodiment a method is used comprising the steps of dispersing calcium; {4-[(5,6-diphenylpyrazin-2-yl)(propan-2-yl)amino]butoxy}acetate, or a pharmaceutically acceptable hydrate or solvate thereof (drug) in a liquid dispersion medium and applying mechanical means in the presence of grinding media to reduce the particle size of the drug to an average effective particle size of 50 μm (micrometer) or less, in particular to the desired particle size distribution Dv50 as indicated above.

The grinding media for the particle size reduction step can be selected from rigid media preferably spherical or particulate in form having an average size less than 3 mm and, more preferably, less than 2 mm, such as 1 mm+10%, or 1 mm+5%. Examples of grinding media are ZrO₂ such as 95% ZrO₂ stabilized with magnesia or stabilized with yttrium, zirconium silicate, glass grinding media, polymeric beads, stainless steel, titania, alumina and the like. Preferred grinding media have a density greater than 2.5 g/cm³ and include 95% ZrO₂ stabilized with magnesia and polymeric beads.

The particles should be reduced in size at a temperature that does not significantly degrade the drug. Processing temperatures of less than 30 to 40° C. are ordinarily preferred. If desired, the processing equipment may be cooled with conventional cooling equipment. The method is conveniently carried out under conditions of ambient temperature and at processing pressures, which are safe and effective for the milling process.

The liquid medium for milling comprises a surfactant/wetting agent, optionally a resuspending agent; and a pharmaceutically acceptable aqueous carrier at a pH in the range of 6 to 8.5, to form a premix/predispersion. The surfactant/wetting agent, the optional resuspending agent, and the pharmaceutically acceptable aqueous carrier, including buffering and pH adjusting agents are preferably those described above. Preferably, the premix/predispersion is over-concentrated, and subsequently diluted to final volume directly before filling.

The final formulation is separated from the grinding media by adequate separation methods known in the field.

The calcium; {4-[(5,6-diphenylpyrazin-2-yl)(propan-2-yl)amino]butoxy}acetate can be sterilized using gamma irradiation, and used for aseptically manufacturing final drug product. The final drug product can be sterilized using gamma irradiation or heat sterilization, e.g. autoclaving (steam sterilising) at elevated temperatures.

Suitable conditions for autoclavation (steam sterilisation) are 15 min at 121-124° C.(±2)° C. A pressure is built up to allow for the desired temperature. Conditions relating to validation as prescribed in the Pharmacopeia, e.g. “US Pharmacopeia”, or “The International Pharmacopoeia, Ninth Edition 2019”, etc. should be taken into account.

Suitable conditions for gamma irradiation are achieved by exposure to ionizing radiation in the form of gamma radiation from a suitable radioisotopic source such as ⁶⁰Co (cobalt 60) or of electrons energized by a suitable electron accelerator. Suitable conditions are radiation levels of 5 to 40 kGy, for instance 5 kGy, 25 kGy or 40 kGy. Conditions relating to validation as prescribed in the Pharmacopeia, e.g. “US Pharmacopeia”, or “The International Pharmacopoeia, Ninth Edition 2019”, etc. should be taken into account.

Hence, the present invention further relates to a process for preparing a sterile pharmaceutical composition as described above, wherein the pharmaceutical composition is sterilized with autoclavation (steam sterilisation), or with gamma-irradiation; or wherein calcium; {4-[(5,6-diphenylpyrazin-2-yl)(propan-2-yl)amino]butoxy}acetate is sterilised with gamma-irradiation and which is then used for preparing the pharmaceutical composition.

A sterile pharmaceutical composition is obtainable by said process.

The bioindicator strain proposed for validation of autoclavation (steam sterilisation) process is: spores of Bacillus stearothermophilus (e.g. ATCC 7953 or CIP 52.81) for which the D-value (i.e. 90% reduction of the microbial population) is 1.5-2 minutes at 121° C., using about 106 spores per indicator.

The bioindicator strains proposed for validation of gamma-irradiation sterilisation process in general are: spores of Bacillus pumilus (e.g. ATCC 27142 or CIP 77.25) with 25 kGy (2.5 Mrad) for which the D-value is about 3 kGy (0.3 Mrad) using 107-108 spores per indicator; for higher doses, spores of Bacillus cereus (e.g. SSI C 1/1) or Bacillus sphaericus (e.g. SSI CIA) are used.

The pharmaceutical compositions as described herein can further be lyophilized, i.e. freeze-dried, and a lyophilized injectable composition will be obtained.

The pharmaceutical composition will, prior to freeze drying, preferably be filled into containers (unit dose or multi-dose containers such as vials) suitable for storage of the lyophilized cake, and suitable for the later reconstitution of the pharmaceutical composition. Such containers may be filled under an inert gas atmosphere (such as notably a nitrogen atmosphere). Such inert gas atmosphere may reduce oxidative degradation of the active ingredient. A further embodiment thus relates to a container such as for example a vial, an ampoule, a syringe, a coupled chamber device, a pen device, or an autoinjector device, especially a vial, filled with a pharmaceutical composition as described above.

The steps for lyophilisation/freeze-drying of the pharmaceutical composition in the form of an aqueous suspension as described herein comprise a step of freezing the pharmaceutical composition in a container, and drying it by applying a vacuum.

The freezing temperature is in the range from −55° C. to −35° C., preferably from −50° C. to −35° C., preferably from −45° C. to −35° C.; for instance −40° C.±3° C.

The drying temperature is in the range from −55° C. to +30° C., preferably from −50° C. to 28° C., preferably from −45° C. to 28° C.

The freezing and the drying temperature may be applied as a fixed temperature, or as a temperature ramp. Preferably, the end temperatures of each procedural step are reached via a temperature ramp.

During freeze-drying, a vacuum is applied to the pharmaceutical composition. Preferably, a vacuum of 0.05 to 1.5 mbar is applied, for instance 0.1 mbar. The vacuum is applied after the freezing step, and during drying.

The drying procedure may be divided into several steps, for instance a primary drying step, and a secondary drying step, whereby each step may be followed by a holding step, i.e. holding the pharmaceutical composition at the temperature and pressure reached at the end of the preceding drying step.

Moreover, the container can be stoppered after the freeze-drying procedure. Stoppering the container may further include a step of capping the container.

The method of freeze-drying preferably comprises the following steps:
a) preparing an aqueous pharmaceutical composition as described above; and
b) freeze-drying said aqueous pharmaceutical composition to form a cake using a method comprising the steps of:
   (i) freezing the aqueous pharmaceutical composition at a first temperature for a period sufficient to transform the liquid formulation into solid state, wherein said first temperature is in the range from −55° C. to −35° C., preferably from −50° C. to −35° C., preferably from −45° C. to −35° C.; for instance to −40° C.±3° C.;
   (ii) optionally holding the frozen composition at the temperature of step (i);
   (iii) applying a primary drying step by subjecting the frozen composition at the temperature of step (i) or (ii) to a vacuum (preferably a vacuum of 0.05 to 1.5 mbar), and applying a temperature ramp in the range from −55° C. to −25° C.; preferably from −50° C. to −25° C., preferably from −45° C. to −25° C., for instance from −40° C.±3° C. to −20° C.±3° C.;
   (iv) optionally holding the frozen composition at the end temperature of step (iii) under vacuum (preferably a vacuum of 0.05 to 1.5 mbar);
   (v) applying a secondary drying step by subjecting the composition of step (iii) or (iv) to a vacuum (preferably a vacuum of 0.05 to 1.5 mbar), and applying a temperature ramp, starting with the end-temperature of step (iii) or step (iv) and proceeding to a temperature in a range from 15° C. to 30° C., preferably from 20° C. to 28° C., for instance 25° C.±3° C.;
   (vi) optionally holding the end-temperature of step (v) and the vacuum;
   (vii) releasing the vacuum.

This method may be applied to an aqueous composition as described above, contained in a container, whereby the container is stoppered, and optionally capped, after releasing of the vacuum.

The term “cake” refers to a dry solid material that results when a liquid formulation has been lyophilized or freeze dried.

The pharmaceutical compositions as described herein can be in the form of a lyophilised pharmaceutical composition. In particular, it can be a lyophilised pharmaceutical composition obtainable by the lyophilisation process described above, e.g. by freezing the pharmaceutical composition in a container, and drying it by applying a vacuum.

Moreover, the lyophilised pharmaceutical composition as described herein may be reconstituted by adding at least one diluent to said lyophilised pharmaceutical composition to provide a reconstituted pharmaceutical composition.

Suitable diluents to reconstitute said pharmaceutical composition include any diluent that is a safe, stable and pharmaceutically acceptable carrier. Preferred is water for injection (WFI) such as especially sterile water for injection (SWFI) or bacteriostatic water for injection (BWFI), optionally containing a tonicity modifier, or mixtures of several tonicity modifiers, such as aqueous (preferably physiological) saline.

One embodiment relates to a pharmaceutical composition as described herein for use in the treatment and/or prevention of a disease and/or disorder selected from the group consisting of ulcer, digital ulcer, diabetic gangrene, diabetic foot ulcer, pressure ulcer (bedsore), hypertension, pulmonary hypertension, pulmonary arterial hypertension, chronic thromboembolic pulmonary hypertension, Fontan disease and pulmonary hypertension associated with Fontan disease, sarcoidosis and pulmonary hypertension associated with sarcoidosis, peripheral circulatory disturbance (e.g., chronic arterial occlusion, intermittent claudication, peripheral embolism, vibration syndrome, Raynaud's disease), connective tissue disease (e.g., systemic lupus erythematosus, scleroderma, mixed connective tissue disease, vasculitic syndrome), reocclusion/restenosis after percutaneous transluminal coronary angioplasty (PTCA), arteriosclerosis, thrombosis (e.g., acute-phase cerebral thrombosis, pulmonary embolism), transient ischemic attack (TIA), diabetic neuropathy, ischemic disorder (e.g., cerebral infarction, myocardial infarction), angina (e.g., stable angina, unstable angina), chronic kidney diseases including glomerulonephritis and diabetic nephropathy at any stage, allergy, bronchial asthma, restenosis after coronary intervention such as atherectomy and stent implantation, thrombocytopenia by dialysis, the diseases in which fibrosis of organs or tissues is involved [e.g., renal diseases such as tubulointerstitial nephritis), respiratory diseases (e.g., interstitial pneumonia, (idiopathic) pulmonary fibrosis, chronic obstructive pulmonary disease), digestive diseases (e.g, hepatocirrhosis, viral hepatitis, chronic pancreatitis and scirrhous stomachic cancer), cardiovascular diseases (e.g, myocardial fibrosis), bone and articular diseases (e.g, bone marrow fibrosis and rheumatoid arthritis), skin diseases (e.g, cicatrix after operation, scalded cicatrix, keloid, and hypertrophic cicatrix), obstetric diseases (e.g., hysteromyoma), urinary diseases (e.g., prostatic hypertrophy), other diseases (e.g., Alzheimer's disease, sclerosing peritonitis, type I diabetes and organ adhesion after operation)], erectile dysfunction (e.g., diabetic erectile dysfunction, psychogenic erectile dysfunction, psychotic erectile dysfunction, erectile dysfunction associated with chronic renal failure, erectile dysfunction after intrapelvic operation for removing prostate, and vascular erectile dysfunction associated with aging and arteriosclerosis), inflammatory bowel disease (e.g., ulcerative colitis, Crohn's disease, intestinal tuberculosis, ischemic colitis and intestinal ulcer associated with Behcet disease), gastritis, gastric ulcer, ischemic ophthalmopathy (e.g., retinal artery occlusion, retinal vein occlusion, ischemic optic neuropathy), sudden hearing loss, avascular necrosis of bone, intestinal damage caused by administration of a non-steroidal anti-inflammatory agent and symptoms associated with lumbar spinal canal stenosis.

Preferred disease and/or disorders are selected from the group consisting of ulcer, digital ulcer, diabetic gangrene, diabetic foot ulcer, pulmonary hypertension, pulmonary arterial hypertension, chronic thromboembolic pulmonary hypertension, Fontan disease and pulmonary hypertension associated with Fontan disease, sarcoidosis and pulmonary hypertension associated with sarcoidosis, peripheral circulatory disturbance, connective tissue disease, chronic kidney diseases including glomerulonephritis and diabetic nephropathy at any stage, diseases in which fibrosis of organs or tissues is involved, and respiratory diseases.

In certain embodiments, the pharmaceutical compositions described herein are for use in the treatment and/or prevention of pulmonary hypertension, in particular, pulmonary arterial hypertension (PAH), chronic thromboembolic pulmonary hypertension, pulmonary hypertension associated with Fontan disease, or pulmonary hypertension associated with sarcoidosis. Particularly preferred is pulmonary arterial hypertension (PAH) or chronic thromboembolic pulmonary hypertension (CTEPH).

The pharmaceutical compositions described herein, in particular for the treatment of the above-indicated diseases and/or disorders, is preferably in the form of an intramuscular or subcutaneous injectable. Thereby, the injectable is a long-acting injectable (LAI). The term “long acting injectable” is used herein for an administration interval of one week to three months, or 1 week to two months, or 1 week to one month, or 1, 2, 3, 4, 5, 6, 7, 8, 9, 10, 11 or 12 weeks.

The pharmaceutical compositions described herein provide release of the active ingredient over a prolonged period of time and therefore it can also be referred to as sustained or delayed release composition. After administration, the composition stays in the body and steadily releases 2-(4-((5,6-diphenylpyrazin-2-yl)(isopropyl)amino)butoxy)acetic acid or its calcium salt, keeping such levels of this active ingredient in the patient's system for a prolonged period of time, thereby providing, during said period, the appropriate treatment or prevention of the above-given diseases and/or disorders, in particular PAH and CTEPH. Because of the fact that the pharmaceutical compositions described herein facilitate that the active ingredient stay in the body and steadily releases the active ingredient, it can be referred to as pharmaceutical composition suitable as long-acting (or depot) formulations.

The pharmaceutical compositions described herein may be applied in the long-term treatment or the long-term prevention of the diseases and/or disorders disclosed herein, in particular PAH and CTEPH.

The pharmaceutical compositions as described herein includes the active ingredient, i.e. calcium; {4-[(5,6-diphenylpyrazin-2-yl)(propan-2-yl)amino]butoxy}acetate (or a pharmaceutically acceptable hydrate or solvate thereof) in a therapeutically effective amount.

The term “therapeutically effective amount” refers to amounts, or concentrations, of the composition (or amounts/concentrations of active ingredient within such composition) that result in efficacious plasma levels for treating the indicated diseases, in particular PAH and CTEPH. For instance, a therapeutically effective amount may be 1 to 200 mg, for example 2 to 150 mg or 5 to 100 mg, and notably 25 mg to 100 mg of calcium; {4-[(5,6-diphenylpyrazin-2-yl)(propan-2-yl)amino]butoxy}acetate per month. With “efficacious plasma levels” it is meant those plasma levels of {4-[(5,6-diphenylpyrazin-2-yl)(propan-2-yl)amino]butoxy}acetic acid, that provide effective treatment or effective prevention of the indicated diseases and/or disorders, in particular PAH and CTEPH.

The dose (or amount) of calcium; {4-[(5,6-diphenylpyrazin-2-yl)(propan-2-yl)amino]butoxy}acetate (or a pharmaceutically acceptable hydrate or solvate thereof) administered also depends on the frequency of the administrations (i.e. the time interval between each administration). Usually, the dose will be higher where administrations are less frequent.

The term “subject” in particular relates to a human being.

The present invention further concerns a method of treating a subject suffering from the above-indicated diseases and/or disorders, in particular PAH and CTEPH, said method comprising the administration of a therapeutically effective amount of a pharmaceutical composition as described herein to a human subject in need thereof. The administration of the present pharmaceutical composition will be via intramuscular or subcutaneous injection.

In particular, the present invention relates to a method for preventing and/or treating ulcer, digital ulcer, diabetic gangrene, diabetic foot ulcer, pulmonary hypertension, pulmonary arterial hypertension, chronic thromboembolic pulmonary hypertension, Fontan disease and pulmonary hypertension associated with Fontan disease, sarcoidosis and pulmonary hypertension associated with sarcoidosis, peripheral circulatory disturbance, connective tissue disease, chronic kidney diseases including glomerulonephritis and diabetic nephropathy at any stage, diseases in which fibrosis of organs or tissues is involved, and respiratory diseases, comprising administering the pharmaceutical compositions as described herein to a human subject in need thereof.

The present invention also concerns the use of calcium; {4-[(5,6-diphenylpyrazin-2-yl)(propan-2-yl)amino]butoxy}acetate of formula (I), or of a pharmaceutically acceptable hydrate or solvate thereof, for the manufacture of a medicament for the treatment of the above-indicated diseases and/or disorders, in particular PAH and CTEPH, said medicament comprising a therapeutically effective amount of calcium; {4-[(5,6-diphenylpyrazin-2-yl)(propan-2-yl)amino]butoxy}acetate of formula (I), or of a pharmaceutically acceptable hydrate or solvate thereof, in the form of an aqueous suspension.

The present invention also concerns calcium; {4-[(5,6-diphenylpyrazin-2-yl)(propan-2-yl)amino]butoxy}acetate of formula (I), or of a pharmaceutically acceptable hydrate or solvate thereof, for use in the treatment of the above-indicated diseases and/or disorders, in particular PAH and CTEPH, wherein said calcium; {4-[(5,6-diphenylpyrazin-2-yl)(propan-2-yl)amino]butoxy}acetate of formula (I), or a pharmaceutically acceptable hydrate or solvate thereof, is within an aqueous suspension.

The present invention also concerns the use of calcium; {4-[(5,6-diphenylpyrazin-2-yl)(propan-2-yl)amino]butoxy}acetate of formula (I), or of a pharmaceutically acceptable hydrate or solvate thereof, for the manufacture of a medicament for the treatment of the above-indicated diseases and/or disorders, in particular PAH and CTEPH, said medicament comprising a therapeutically effective amount of calcium; {4-[(5,6-diphenylpyrazin-2-yl)(propan-2-yl)amino]butoxy}acetate of formula (I), or of a pharmaceutically acceptable hydrate or solvate thereof, in the form of a lyophilized cake.

The present invention also concerns calcium; {4-[(5,6-diphenylpyrazin-2-yl)(propan-2-yl)amino]butoxy}acetate of formula (I), or of a pharmaceutically acceptable hydrate or solvate thereof, for use in the treatment of the above-indicated diseases and/or disorders, in particular PAH and CTEPH, wherein said calcium; {4-[(5,6-diphenylpyrazin-2-yl)(propan-2-yl)amino]butoxy}acetate of formula (I), or a pharmaceutically acceptable hydrate or solvate thereof, is in the form of a lyophilized cake.

The present invention also concerns the use of calcium; {4-[(5,6-diphenylpyrazin-2-yl)(propan-2-yl)amino]butoxy}acetate of formula (I), or of a pharmaceutically acceptable hydrate or solvate thereof, for the manufacture of a medicament for the treatment of the above-indicated diseases and/or disorders, in particular PAH and CTEPH, said medicament comprising a therapeutically effective amount of calcium; {4-[(5,6-diphenylpyrazin-2-yl)(propan-2-yl)amino]butoxy}acetate of formula (I), or of a pharmaceutically acceptable hydrate or solvate thereof having a particle size distribution Dv50 of 1 to 50 μm (micrometer), preferably 2 to 30 μm or 2 to 20 μm or 5 to 15 μm; a surfactant and/or wetting agent; and a pharmaceutically acceptable aqueous carrier at a pH in the range of 6 to 8.5, in the form of an aqueous suspension.

The present invention also concerns calcium; {4-[(5,6-diphenylpyrazin-2-yl)(propan-2-yl)amino]butoxy}acetate of formula (I), or of a pharmaceutically acceptable hydrate or solvate thereof, for use in the treatment of the above-indicated diseases and/or disorders, in particular PAH and CTEPH, wherein said calcium; {4-[(5,6-diphenylpyrazin-2-yl)(propan-2-yl)amino]butoxy}acetate of formula (I), or a pharmaceutically acceptable hydrate or solvate thereof having a particle size distribution Dv50 of 1 to 50 μm (micrometer), preferably 2 to 30 μm or 2 to 20 μm or 5 to 15 μm; a surfactant and/or wetting agent; and a pharmaceutically acceptable aqueous carrier at a pH in the range of 6 to 8.5, is within an aqueous suspension.

The present invention also concerns the use of calcium; {4-[(5,6-diphenylpyrazin-2-yl)(propan-2-yl)amino]butoxy}acetate of formula (I), or of a pharmaceutically acceptable hydrate or solvate thereof, for the manufacture of a medicament for the treatment of the above-indicated diseases and/or disorders, in particular PAH and CTEPH, said medicament comprising a therapeutically effective amount of calcium; {4-[(5,6-diphenylpyrazin-2-yl)(propan-2-yl)amino]butoxy}acetate of formula (I), or of a pharmaceutically acceptable hydrate or solvate thereof, in the form of an intramuscular or subcutaneous injectable.

The present invention also concerns calcium; {4-[(5,6-diphenylpyrazin-2-yl)(propan-2-yl)amino]butoxy}acetate of formula (I), or of a pharmaceutically acceptable hydrate or solvate thereof, for use in the treatment of the above-indicated diseases and/or disorders, in particular PAH and CTEPH, wherein said calcium; {4-[(5,6-diphenylpyrazin-2-yl)(propan-2-yl)amino]butoxy}acetate of formula (I), or a pharmaceutically acceptable hydrate or solvate thereof, is in the form of an intramuscular or subcutaneous injectable.

The present invention also concerns the use of calcium; {4-[(5,6-diphenylpyrazin-2-yl)(propan-2-yl)amino]butoxy}acetate of formula (I), or of a pharmaceutically acceptable hydrate or solvate thereof, for the manufacture of a medicament for the treatment of the above-indicated diseases and/or disorders, in particular PAH and CTEPH, said medicament comprising a therapeutically effective amount of calcium; {4-[(5,6-diphenylpyrazin-2-yl)(propan-2-yl)amino]butoxy}acetate of formula (I), or of a pharmaceutically acceptable hydrate or solvate thereof, wherein said medicament is administered at a time interval of one week to three months, preferably one week or one month or three months.

The present invention also concerns calcium; {4-[(5,6-diphenylpyrazin-2-yl)(propan-2-yl)amino]butoxy}acetate of formula (I), or of a pharmaceutically acceptable hydrate or solvate thereof, for use in the treatment of the above-indicated diseases and/or disorders, in particular PAH and CTEPH, wherein said calcium; {4-[(5,6-diphenylpyrazin-2-yl)(propan-2-yl)amino]butoxy}acetate of formula (I), or a pharmaceutically acceptable hydrate or solvate thereof, wherein it is administered at a time interval of one week to three months, preferably one week or one month or three months.

The present invention further concerns a pharmaceutical composition for use as a long acting injectable in the treatment of and/or prevention of pulmonary hypertension, wherein the pharmaceutical composition is in the form of an aqueous suspension comprising calcium; {4-[(5,6-diphenylpyrazin-2-yl)(propan-2-yl)amino]butoxy}acetate of formula (I), or a pharmaceutically acceptable hydrate or solvate thereof:

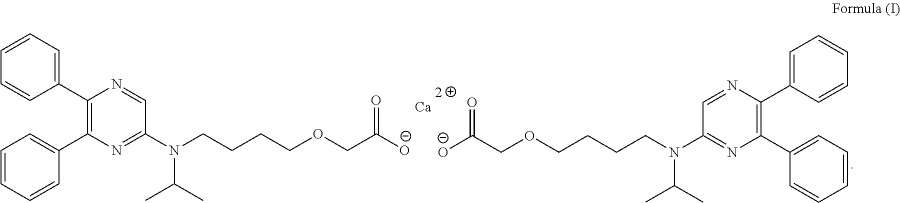

In particular, said pharmaceutical composition for use as a long acting injectable will be for the treatment of and/or prevention of pulmonary arterial hypertension, chronic thromboembolic pulmonary hypertension, pulmonary hypertension associated with Fontan disease, or pulmonary hypertension associated with sarcoidosis. Said pharmaceutical composition for use as a long acting injectable may notably be for use in the treatment and/or prevention of pulmonary arterial hypertension (PAH). Said pharmaceutical composition for use as a long acting injectable may also be for use in the treatment and/or prevention of chronic thromboembolic pulmonary hypertension (CTEPH). Said pharmaceutical composition for the previously mentioned uses may be in the form of an intramuscular or subcutaneous injectable. In particular, said intramuscular or subcutaneous injectable may be administered at a time interval of one week to three months, notably at a time interval of two weeks to one month. The suspended particles of said intramuscular or subcutaneous injectable may have a particle size distribution Dv50 of 1 to 50 μm (micrometer), preferably 2 to 30 μm or 2 to 20 μm or 5 to 15 μm.

The present invention further relates to calcium; {4-[(5,6-diphenylpyrazin-2-yl)(propan-2-yl)amino]butoxy}acetate of formula (I), or a pharmaceutically acceptable hydrate or solvate thereof

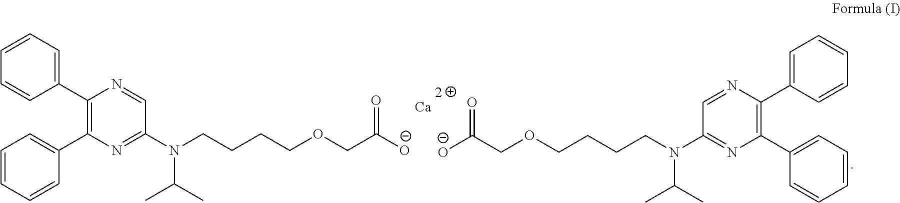

having a particle size distribution Dv50 of 1 to 50 μm (micrometer), preferably 2 to 30 μm or 2 to 20 μm or 5 to 15 μm.

The present invention further relates to calcium; {4-[(5,6-diphenylpyrazin-2-yl)(propan-2-yl)amino]butoxy}acetate of formula (I), or a pharmaceutically acceptable hydrate or solvate thereof

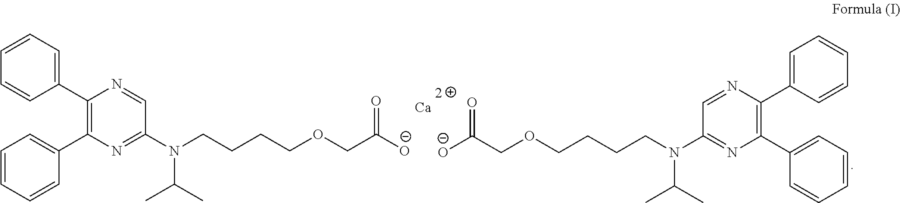

having a particle size distribution Dv50 of 1 to 50 μm (micrometer), preferably 2 to 30 μm or 2 to 20 μm or 5 to 15 μm, wherein said particles are suspended in an aqueous medium. Said aqueous medium, in addition to water, may comprise (i) a surfactant and/or wetting agent; and optionally (ii) a resuspending agent. Furthermore, the pH of said aqueous medium may be in the range of 6 to 9, and in particular in the range of 6 to 8.5.

Moreover, the present invention relates to calcium; {4-[(5,6-diphenylpyrazin-2-yl)(propan-2-yl)amino]butoxy}acetate of formula (I), or a pharmaceutically acceptable hydrate or solvate thereof

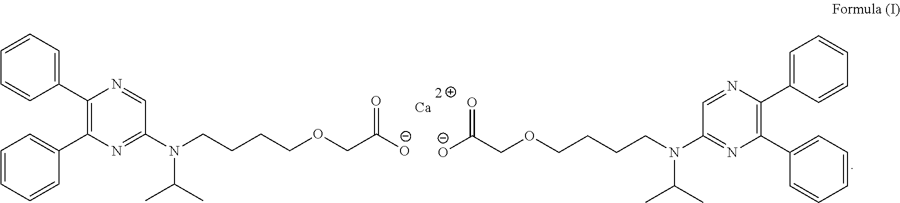

having a particle size distribution Dv50 of 1 to 50 μm (micrometer), preferably 2 to 30 μm or 2 to 20 μm or 5 to 15 μm, wherein said particles are suspended in an aqueous medium, for use in the treatment of a disease and/or disorder selected from the group consisting of ulcer, digital ulcer, diabetic gangrene, diabetic foot ulcer, pressure ulcer (bedsore), hypertension, pulmonary hypertension, pulmonary arterial hypertension, chronic thromboembolic pulmonary hypertension, Fontan disease and pulmonary hypertension associated with Fontan disease, sarcoidosis and pulmonary hypertension associated with sarcoidosis, peripheral circulatory disturbance (e.g., chronic arterial occlusion, intermittent claudication, peripheral embolism, vibration syndrome, Raynaud's disease), connective tissue disease (e.g., systemic lupus erythematosus, scleroderma, mixed connective tissue disease, vasculitic syndrome), reocclusion/restenosis after percutaneous transluminal coronary angioplasty (PTCA), arteriosclerosis, thrombosis (e.g., acute-phase cerebral thrombosis, pulmonary embolism), transient ischemic attack (TIA), diabetic neuropathy, ischemic disorder (e.g., cerebral infarction, myocardial infarction), angina (e.g., stable angina, unstable angina), chronic kidney diseases including glomerulonephritis and diabetic nephropathy at any stage, allergy, bronchial asthma, restenosis after coronary intervention such as atherectomy and stent implantation, thrombocytopenia by dialysis, the diseases in which fibrosis of organs or tissues is involved [e.g., renal diseases such as tubulointerstitial nephritis), respiratory diseases (e.g., interstitial pneumonia, (idiopathic) pulmonary fibrosis, chronic obstructive pulmonary disease), digestive diseases (e.g, hepatocirrhosis, viral hepatitis, chronic pancreatitis and scirrhous stomachic cancer), cardiovascular diseases (e.g, myocardial fibrosis), bone and articular diseases (e.g, bone marrow fibrosis and rheumatoid arthritis), skin diseases (e.g, cicatrix after operation, scalded cicatrix, keloid, and hypertrophic cicatrix), obstetric diseases (e.g., hysteromyoma), urinary diseases (e.g., prostatic hypertrophy), other diseases (e.g., Alzheimer's disease, sclerosing peritonitis, type I diabetes and organ adhesion after operation)], erectile dysfunction (e.g., diabetic erectile dysfunction, psychogenic erectile dysfunction, psychotic erectile dysfunction, erectile dysfunction associated with chronic renal failure, erectile dysfunction after intrapelvic operation for removing prostate, and vascular erectile dysfunction associated with aging and arteriosclerosis), inflammatory bowel disease (e.g., ulcerative colitis, Crohn's disease, intestinal tuberculosis, ischemic colitis and intestinal ulcer associated with Behcet disease), gastritis, gastric ulcer, ischemic ophthalmopathy (e.g., retinal artery occlusion, retinal vein occlusion, ischemic optic neuropathy), sudden hearing loss, avascular necrosis of bone, intestinal damage caused by administration of a non-steroidal anti-inflammatory agent and symptoms associated with lumbar spinal canal stenosis; in particular pulmonary hypertension and specially a disease and/or disorder selected from the group consisting of PAH and CTEPH. Said aqueous medium, in addition to water, may comprise (i) a surfactant and/or wetting agent; and optionally (ii) a resuspending agent. Furthermore, the pH of said aqueous medium may be in the range of 6 to 9, and in particular in the range of 6 to 8.5.

In particular, the present invention relates to calcium; {4-[(5,6-diphenylpyrazin-2-yl)(propan-2-yl)amino]butoxy}acetate of formula (I), or a pharmaceutically acceptable hydrate or solvate thereof

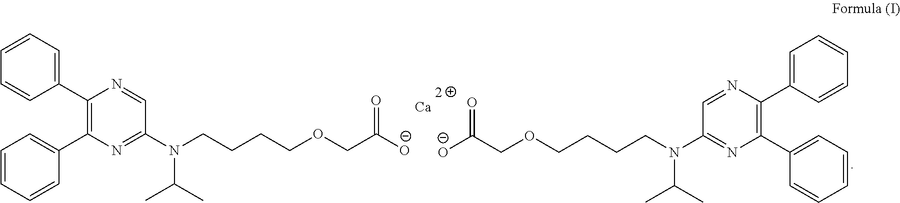

having a particle size distribution Dv50 of 1 to 50 μm (micrometer), preferably 2 to 30 μm or 2 to 20 μm or 5 to 15 μm, wherein said particles are suspended in an aqueous medium, for use in the treatment of pulmonary hypertension, especially PAH or CTEPH, wherein said particles suspended in said aqueous medium are for administration by intramuscular or subcutaneous injection. Said aqueous medium, in addition to water, may comprise (i) a surfactant and/or wetting agent; and optionally (ii) a resuspending agent. Furthermore, the pH of said aqueous medium may be in the range of 6 to 9, and in particular in the range of 6 to 8.5. In particular, said intramuscular or subcutaneous injection is for administration at a time interval of one week to three months, notably at a time interval of two weeks to one month.

Finally, the invention also relates to an investigational drug (‘ID’) in the form of an aqueous suspension comprising calcium; {4-[(5,6-diphenylpyrazin-2-yl)(propan-2-yl)amino]butoxy}acetate of formula (I), or a pharmaceutically acceptable hydrate or solvate thereof:

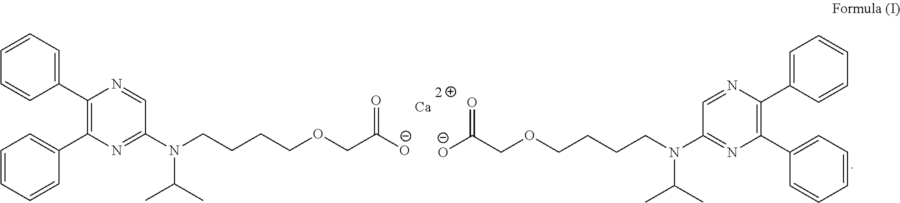

By “Investigational New Drug” or “investigational drug” is meant herein a new drug or biological drug that is used in a clinical investigation. Preferably, the investigational drug will be used in a clinical investigation regarding the treatment of pulmonary hypertension, in particular PAH or CTEPH.

According to one embodiment, said ID will be safe and efficacious for the treatment of and/or prevention of pulmonary arterial hypertension, chronic thromboembolic pulmonary hypertension, pulmonary hypertension associated with Fontan disease, or pulmonary hypertension associated with sarcoidosis, notably for the treatment of pulmonary hypertension and in particular the treatment of PAH or CTEPH. Said ID for the previously mentioned uses may be in the form of an intramuscular or subcutaneous injectable. In particular, said intramuscular or subcutaneous injectable may be administered at a time interval of one week to three months, notably at a time interval of two weeks to one month. The suspended particles of said intramuscular or subcutaneous injectable may have a particle size distribution Dv50 of 1 to 50 μm (micrometer), preferably 2 to 30 μm or 2 to 20 μm or 5 to 15 μm.

The present invention further relates to an ID in the form of an aqueous suspension comprising calcium; {4-[(5,6-diphenylpyrazin-2-yl)(propan-2-yl)amino]butoxy}acetate of formula (I), or a pharmaceutically acceptable hydrate or solvate thereof

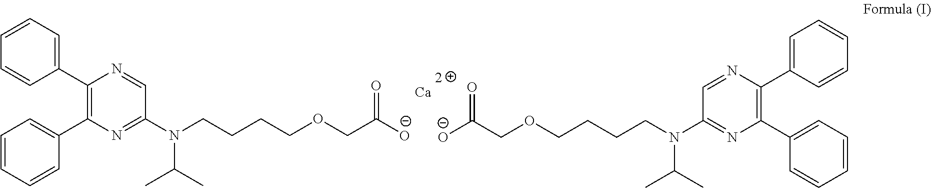

having a particle size distribution Dv50 of 1 to 50 μm (micrometer), preferably 2 to 30 μm or 2 to 20 μm or 5 to 15 μm.

The present invention further relates to an ID in the form of an aqueous suspension comprising calcium; {4-[(5,6-diphenylpyrazin-2-yl)(propan-2-yl)amino]butoxy}acetate of formula (I), or a pharmaceutically acceptable hydrate or solvate thereof

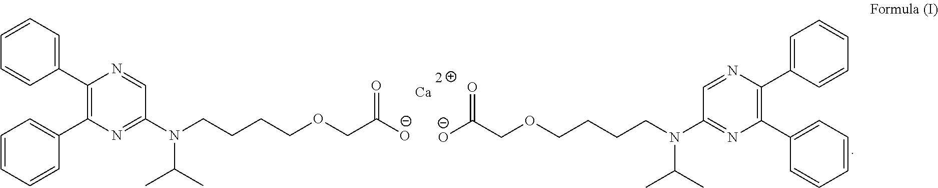

having a particle size distribution Dv50 of 1 to 50 μm (micrometer), preferably 2 to 30 μm or 2 to 20 μm or 5 to 15 μm, wherein said particles are suspended in an aqueous medium. Said aqueous medium, in addition to water, may comprise (i) a surfactant and/or wetting agent; and optionally (ii) a resuspending agent. Furthermore, the pH of said aqueous medium may be in the range of 6 to 9, and in particular in the range of 6 to 8.5.

In particular, the present invention relates to an ID in the form of an aqueous suspension comprising calcium; {4-[(5,6-diphenylpyrazin-2-yl)(propan-2-yl)amino]butoxy}acetate of formula (I), or a pharmaceutically acceptable hydrate or solvate thereof

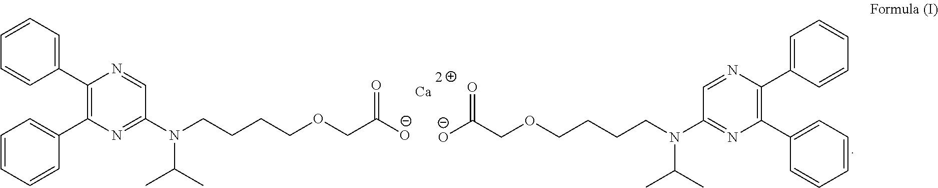

having a particle size distribution Dv50 of 1 to 50 μm (micrometer), preferably 2 to 30 μm or 2 to 20 μm or 5 to 15 μm, wherein said particles are suspended in an aqueous medium, for use in the treatment of pulmonary hypertension, especially PAH or CTEPH, wherein said particles suspended in said aqueous medium are for administration by intramuscular or subcutaneous injection. Said aqueous medium, in addition to water, may comprise (i) a surfactant and/or wetting agent; and optionally (ii) a resuspending agent. Furthermore, the pH of said aqueous medium may be in the range of 6 to 9, and in particular in the range of 6 to 8.5. In particular, said intramuscular or subcutaneous injection is for administration at a time interval of one week to three months, notably at a time interval of two weeks to one month.

All documents cited herein are incorporated by reference in their entirety.

The following examples are intended to illustrate the present invention and should not be construed as limiting the invention thereto.

### EXAMPLES

### Abbreviations (as Used Herein and in the Description Above)

ADME absorption, distribution, metabolism, and excretion
API Active Pharmaceutical Ingredient
aq. aqueous
BHA butylated hydroxyanisole
BHT butylated hydroxytoluene
CTAC hexyldecyl trimethyl ammonium chloride
EDTA ethylenediaminetetraacetic acid
HPLC high performance liquid chromatography
IM intramuscular
INCI international nomenclature of cosmetic ingredients
INN international nonproprietary name
IP receptor prostacyclin receptor
ISO International Organization of Standardization
LAI long acting injectable
LD laser diffraction
min minute(s)
mM millimole
NMP N-methylpyrrolidone
PEG polyethylene glycol
PSD particle size distribution
PAH Pulmonary Arterial Hypertension
CTEPH chronic thromboembolic pulmonary hypertension
PBS Phosphate Buffered Saline
pK pharmacokinetic
PVP polyvinylpyrrolidone
q.s. quantum satis (as much as is sufficient)
q.s. ad quantum satis (as much as is sufficient) to make
RT room temperature
SC subcutaneous
SDS sodium dodecyl sulphate
TRIS tris(hydroxymethyl)aminomethane
UPLC Ultra performance liquid chromatography
WFI water for injection
WHO World Health Organization w/v weight per volume w/w weight per weight XRPD X-ray powder diffraction

### PSD Measurement

PSD was measured with a Malvern Mastersizer 3000 apparatus from Malvern Panalytical using the laser diffraction measurement method and the Mie theory. The results of the laser diffraction analysis are reported based on the particle size volume distribution as the cumulative undersize values dv50. The following settings were used:

Particle Type

| | |
|---|---|
| Non-spherical particle mode | Yes |
| Is Fraunhofer type | No |

Material properties

| Material name | Mie 1.63/0.01 | |
|---|---|---|
| Refractive index | 1.630 | |
| Absorption index | 0.010 | |
| Particle density | 1.00 | g/cm³ |

| | |
|---|---|
| Different optical properties in blue | No |
| light | |

Dispersant properties

| | |
|---|---|
| Dispersant name | Water |
| Refractive index | 1.330 |
| Level sensor threshold | 100.000 |

Measurement duration

| Background measurement | 15.00 | s |
|---|---|---|
| duration (red) | | |
| Sample measurement duration | 15.00 | s |
| (red) | | |
| Perform blue light measurement? | Yes | |
| Background measurement | 15.00 | s |
| duration (blue) | | |
| Sample measurement duration | 15.00 | s |

| | |
|---|---|
| (blue) | |
| Assess light background stability | No |

Measurement sequence

| Aliquots | 1 | |
|---|---|---|
| Automatic number of | No | |
| measurements | | |
| Pre-alignment delay | 0.00 | s |
| Number of measurements | 3 | |
| Delay between measurements | 0.00 | s |
| Pre-measurement delay | 60.00 | s |

| | |
|---|---|
| Close measurement window after | No |
| measurement | |

Measurement obscuration settings

| | |
|---|---|
| Auto start measurement | No |
| Obscuration low limit | 4.00% |
| Obscuration high limit | 6.00% |
| Enable obscuration filtering | No |

Measurement alarms

| | |
|---|---|
| Use Background Check | No |
| Background Check Limits | [1; 200]; [20; 60] |

Accessory control settings

| Accessory name | Hydro MV | |
|---|---|---|
| Is accessory dry? | No | |
| Stirrer speed | 1750 | rpm |

| | |
|---|---|
| Ultrasound percentage | 50% |
| Fill Dispersant Source Identifier | Manual |
| Manual tank fill? | Yes |
| Degas after tank and cell fill | Yes |
| Sonicate to stability? | No |
| Ultrasound mode | None |

Post-Measurement clean sequence settings

| | |
|---|---|
| Clean sequence type | None |
| Sonicate during clean? | No |
| Manually Fill Tank During Clean? | No |
| Clean Dispersant Source | Auto |
| Identifier | |
| Clean Dispersant Level Sensor | 0 |
| Threshold | |
| Degas After Clean? | Yes |
| Drain Valve Flush? | Yes |
| Tank Overfill? | Yes |

Analysis settings

| | |
|---|---|
| Analysis model | General Purpose |
| Single result mode | No |
| Number of excluded inner | 0 |
| detectors | |
| Blue light detectors excluded | No |
| Fine powder mode | No |
| Analysis sensitivity | Normal |
| Analysed as Mastersizer 3000E? | No |

Result Settings

| | |
|---|---|
| Result range is limited | No |
| Result Units | Volume |
| Extend Result | No |
| Result Emulation | No |

User sizes for histograms and tables

| | |
|---|---|
| Use user sizes | No |

Data export output

| | |
|---|---|
| Enabled? | No |

Averaging

| | |
|---|---|
| Averaging enabled? | Yes |

Printing options

| | |
|---|---|
| Printing enabled? | No |

Software used: Mastersizer software v3.81; Malvern Instruments Ltd

Software used: Mastersizer software v3.81; Malvern Instruments Ltd

### Examples

### Example 1: Preparation of calcium; {4-[(5,6-diphenylpyrazin-2-yl)(propan-2-yl)amino]butoxy}acetate

12 g (28.604 mmol) of {4-[(5,6-diphenylpyrazin-2-yl)(propan-2-yl)amino]butoxy}acetic acid were added to a two piece 400 ml reactor and 145.34 g of acetone/water (95/5% w/w) were added. Ramp stirring to a speed of 400 rpm was applied, and the reactor was heated to 50° C. at 1 K/min, and kept at that temperature for 30 min. Then, 15 vol % (4.2 ml) of Ca(OAc)₂×½H₂O dissolved in water (stock solution containing 2.51 g (15.012 mmol) Ca(OAc)₂×½H₂O in 26.66 g water)) were added over 30 min. The mixture was kept for 8 h. Then, the rest of the stock solution of Ca(OAc)₂ dissolved in water was added over 2 h. The mixture was stirred for 7.75 h, and the obtained solid was filtered off, washed with 24 g (2 g/g) acetone/water 80/20% w/w at 50° C. After drying at 50° C. under vacuum and N₂ purge, 12.46 g of calcium; {4-[(5,6-diphenylpyrazin-2-yl)(propan-2-yl)amino]-butoxy}acetate (99.3%) were obtained as crystalline solid.

### Example 2: Feasibility pK Rat Study

An initial pK rat study was conducted to demonstrate the LAI potential of an aqueous micro-suspension of calcium; {4-[(5,6-diphenylpyrazin-2-yl)(propan-2-yl)amino]butoxy}acetate. For this study, aqueous micro-suspensions of Selexipag, {4-[(5,6-diphenylpyrazin-2-yl)(propan-2-yl)amino]butoxy}acetic acid and calcium; {4-[(5,6-diphenylpyrazin-2-yl)(propan-2-yl)amino]butoxy}acetate were prepared. An overview of the design of the study can be found in Table 1.

TABLE 1
pK rat study design (ADME) to demonstrate LAI feasibility

| | | | API eq. | Particle | | |
|---|---|---|---|---|---|---|
| | | | concentration | size | Injection | Dose |
| Group | Formulation | API | (mg/mL) | (Dv50, μm) | route | (mg/kg) |
| F | PVP K17 | Selexipag | 125 | 6.6 | IM | 50 |
| | Citrate buffer | | | | | |
| | pH 5 | | | | | |
| G | PVP K17 | Ca-salt of | 125 | 3.5 | IM | 50 |
| | Citrate buffer | ACT-333679 | | | | |
| | pH 8 | | | | | |
| H | PVP K17 | ACT-333679 | 125 | 4.3 | IM | 50 |
| | Citrate buffer | | | | | |
| | pH 5 | | | | | |

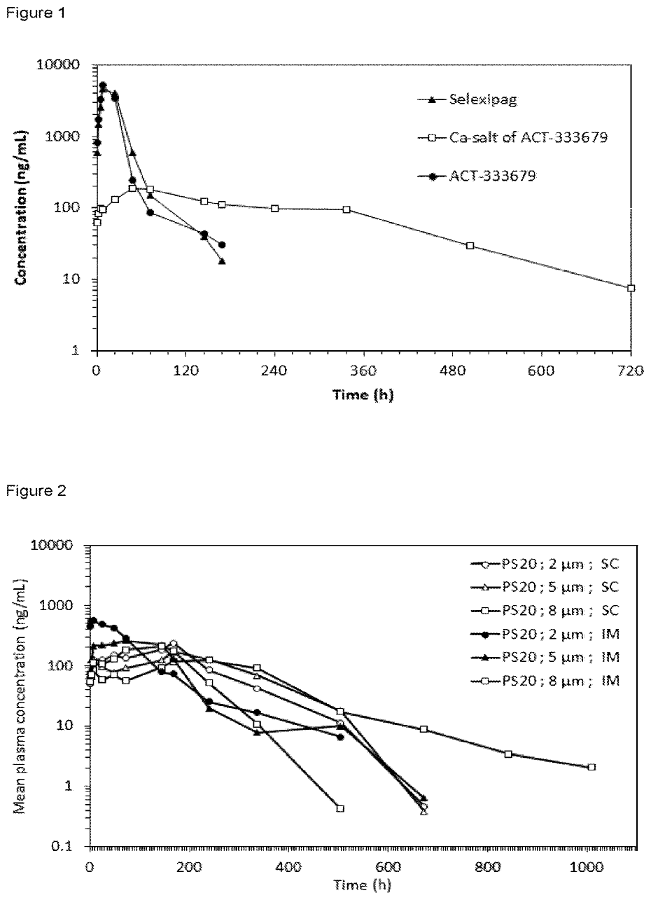

Release profiles and mean AUC of the different formulations are depicted in FIG. 1.

As shown in FIG. 1, the study group dosed with the Ca-salt of ACT-333679 exhibits significant lower plasma concentrations compared to both other groups, which demonstrates a long-acting release profile up to 336 hours (i.e. 14 days) and the AUC increases up until 720 hours. Selexipag and its metabolite (group F and H) did not demonstrate a long-acting release profile because of their high solubility and dissolution rate.

### Example 3: pK Rat Study Comparing Particle Size and Surfactants/Wetting Agents

A pK rat study was set up to evaluate the effect of Ca-salt of ACT-333679 physical properties (i.e. PSD), surfactant/wetting agent and administration route on the in-vivo drug release rate. Particle size of the calcium; {4-[(5,6-diphenylpyrazin-2-yl)(propan-2-yl)amino]butoxy}acetate at Dv50 was varied between 2, 5 and 8 μm. The API concentration of the different studied aqueous suspensions was kept constant at 100 mg/mL eq. Both intramuscular and subcutaneous injection routes were investigated in this rat study. An overview of the studied groups is shown in Table 2.

TABLE 2
Design overview of pK rat study Ex. 3

| | | API eq. | | | Dose | Dose |
|---|---|---|---|---|---|---|
| | | concentration | Particle size | Injection | (mg- | volume, |
| Group* | Formulation | (mg/mL) | (Dv50, μm) | route | eq./kg) | (mL/kg) |
| 1 | Immediate Release (IR) | 10 | N/A | SC | 4.0 | 0.4 |
| | solution | | | | | |
| | 100% PEG 400 | | | | | |
| 2 | Immediate Release (IR) | 10 | N/A | IM | 4.0 | 0.4 |
| | solution | | | | | |
| | 100% PEG 400 | | | | | |
| 3 | 5 mg/mL Polysorbate 20 | 100 | 2 | SC | 40.0 | 0.4 |
| | 50 mg/mL PEG 4000 | | | | | |
| | 50 mmol Tris (pH 8) | | | | | |
| 4 | 5 mg/mL Polysorbate 20 | 100 | 2 | IM | 40.0 | 0.4 |
| | 50 mg/mL PEG 4000 | | | | | |
| | 50 mmol Tris (pH 8) | | | | | |
| 5 | 5 mg/mL Polysorbate 20 | 100 | 5 | SC | 40.0 | 0.4 |
| | 50 mg/mL PEG 4000 | | | | | |
| | 50 mmol Tris (pH 8) | | | | | |
| 6 | 5 mg/mL Polysorbate 20 | 100 | 5 | IM | 40.0 | 0.4 |
| | 50 mg/mL PEG 4000 | | | | | |
| | 50 mmol Tris (pH 8) | | | | | |
| 7 | 5 mg/mL Polysorbate 20 | 100 | 8 | SC | 40.0 | 0.4 |
| | 50 mg/mL PEG 4000 | | | | | |
| | 50 mmol Tris (pH 8) | | | | | |
| 8 | 5 mg/mL Polysorbate 20 | 100 | 8 | IM | 40.0 | 0.4 |
| | 50 mg/mL PEG 4000 | | | | | |
| | 50 mmol Tris (pH 8) | | | | | |
| 9 | 15 mg/mL Poloxamer 338 | 100 | 2 | SC | 40.0 | 0.4 |
| | 50 mg/mL PEG 4000 | | | | | |
| | 50 mmol Tris (pH 8) | | | | | |
| 10 | 15 mg/mL Poloxamer 338 | 100 | 2 | IM | 40.0 | 0.4 |
| | 50 mg/mL PEG 4000 | | | | | |
| | 50 mmol Tris (pH 8) | | | | | |
| 11 | 15 mg/mL Poloxamer 338 | 100 | 5 | SC | 40.0 | 0.4 |
| | 50 mg/mL PEG 4000 | | | | | |
| | 50 mmol Tris (pH 8) | | | | | |
| 12 | 15 mg/mL Poloxamer 338 | 100 | 5 | IM | 40.0 | 0.4 |
| | 50 mg/mL PEG 4000 | | | | | |
| | 50 mmol Tris (pH 8) | | | | | |
| 13 | 15 mg/mL Poloxamer 338 | 100 | 8 | SC | 40.0 | 0.4 |
| | 50 mg/mL PEG 4000 | | | | | |
| | 50 mmol Tris (pH 8) | | | | | |
| 14 | 15 mg/mL Poloxamer 338 | 100 | 8 | IM | 40.0 | 0.4 |
| | 50 mg/mL PEG 4000 | | | | | |
| | 50 mmol Tris (pH 8) | | | | | |

*N = 4 male rats per group

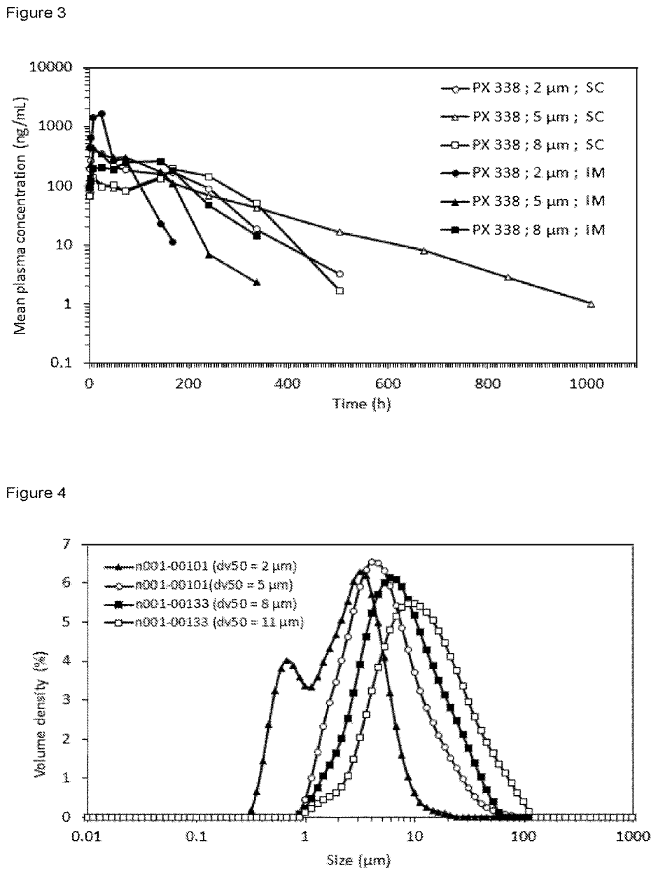

An overview of pK profiles for the different studied groups are visualized in FIGS. 2 and 3. It can be concluded that a long acting extended in vivo release profiles were observed in the whole range from Dv50 from 2 μm to 8 μm. The larger the particle size, the lower the initial release and the longer the duration time was observed. To reach 1-month (or longer) drug release, particle size at Dv50 should be equal or bigger than 8 μm.

### Example 4: Preparation of Formulation Examples—Particle Size

4 separate vials (volume=50 mL) were prepared with varying target particle sizes (Dv50 of 2, 5, 8 and 12 μm (micrometer)). In each vial, 1.568 g (100 mg/mL eq.) of calcium; {4-[(5,6-diphenylpyrazin-2-yl)(propan-2-yl)amino]butoxy}acetate was weighed. Next to the active ingredient, 45 g of 1 mm Zirconium beads was added to each vial. In a next step 12 mL of a 125% over-concentrated stock solution of Polysorbate 20, PEG 4000 and buffer was added to each vial. The compositions of the stock solutions were dependent on the experiment. The composition of each stock solution is shown in Table 3 below.

TABLE 3
Composition of the tested 125% over-concentrated
Polysorbate 20/PEG 4000/buffer (pH 8) stock solutions

| | Concentration (mg/mL) | |
|---|---|---|
| | Experimental ID | |
| Ingredient | n001-00101 | n001-00133 |
| Tween 20 (polysorbate 20) | 6.25 | |
| PEG 4000 | 62.5 | 93.75 |
| Disodium hydrogen | N/A | 10.00 |
| phosphate anhydrous | | |
| Citric acid monohydrate | N/A | 0.2125 |
| Trometamol (TRIS) | 7.571 | N/A |

| | |
|---|---|
| 1N HCl | q.s. ad pH 8 |
| Purified water | q.s. |

In a last step, 3 mL of purified water was added to each vial. All 4 vials were put on a roller mill with a rotational speed of 300 rpm. To reach different particle sizes (Dv50 of 2, 5, 8 and 12 μm), different milling times were needed. An overview of the different milling times and the resulting particle sizes (obtained with Mastersizer® 3000) is shown in Table 4.

TABLE 4
Overview of the different milling
times and the resulting particle sizes.

| Experimental | Milling | | Particle size | | |
|---|---|---|---|---|---|
| ID | time | | (Dv10, μm) | (Dv50, μm) | (Dv90, μm) |
| n001-00101 | 23 | hours | 0.6 | 2.2 | 5.8 |
| | 90 | min. | 2.0 | 5.1 | 16.3 |
| n001-00133 | 16 | min. | 2.9 | 8.0 | 26.1 |
| | 2 | min. | 3.9 | 11.3 | 37.3 |

Each milled suspension was harvested in 8 mL vials and final particle size was determined with Mastersizer® 3000. An overlap of the resulting particle size distributions is shown in FIG. 4.

### Example 5: Preparation of Formulation Examples—Surfactant/Wetting Agent

3 separate vials (volume=50 mL) were prepared with different surfactants/wetting agents (Polysorbate 20, Poloxamer 338, and Vitamin E TPGS). In each vial, 1.568 g (100 mg/mL eq.) of calcium; {4-[(5,6-diphenylpyrazin-2-yl)(propan-2-yl)amino]butoxy}acetate was weighed. Next to the active ingredient, 45 g of 1 mm Zirconium beads was added to each vial. In a next step 12 mL of a 125% over-concentrated stock solution of surfactant/wetting agent, PEG 4000 and Mcllvaine buffer was added to each vial. The composition of the stock solution was dependent on the experiment. The composition of each stock solution is shown in Table 24 below.

TABLE 5
Composition of the tested 125% over-concentrated surfactant (and/or
wetting agent)/PEG 4000/McIlvaine buffer (pH 8) stock solutions

| | Concentration (mg/mL) | | |
|---|---|---|---|
| | Experimental ID | | |
| Ingredient | n001-00119 | | |
| Polysorbate 20 | 6.25 | N/A | N/A |
| Poloxamer 338 | N/A | 18.75 | N/A |
| Vitamin E TPGS | N/A | N/A | 6.25 |

| | |
|---|---|
| PEG 4000 | 93.75 |
| Disodium hydrogen | 10.00 |
| phosphate anhydrous | |
| Citric acid monohydrate | 0.2125 |
| 1N HCl | q.s. ad pH 8 |
| Purified water | q.s. |

In a last step, 3 mL of purified water was added to each vial. All 3 vials were put on a roller mill with a rotational speed of 300 rpm. To reach a target particle size Dv50 of 8 μm, different milling times were needed for the different surfactants/wetting agents. An overview of the different milling times and the resulting particle sizes (obtained with Mastersizer® 3000) is shown in Table 6.

TABLE 6
Overview of the different milling
times and the resulting particle sizes.

| Surfactant/ | Milling | Particle size | | |
|---|---|---|---|---|
| wetting agent | time | (Dv10, μm) | (Dv50, μm) | (Dv90, μm) |
| Polysorbate 20 | 32 min. | 2.9 | 8.0 | 29.4 |
| Poloxamer 338 | 27 min. | 2.9 | 8.0 | 29.6 |
| Vitamin E TPGS | 27 min. | 2.9 | 8.1 | 29.9 |

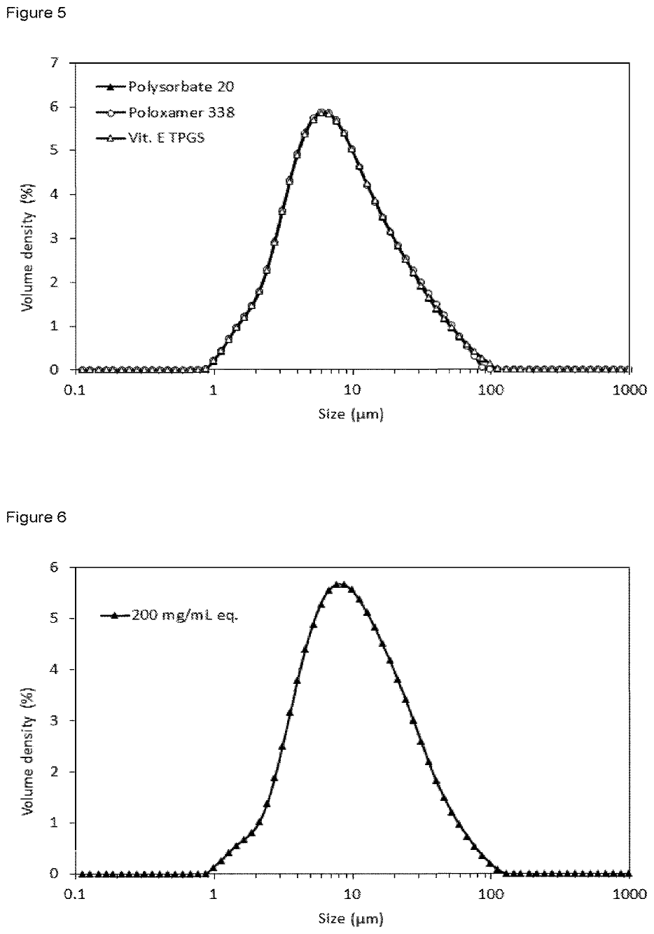

Each milled suspension was harvested in both 8 mL vials and pre-filled syringes. Final particle size was determined with Mastersizer® 3000. An overlap of the resulting particle size distributions is shown in FIG. 5.

All resulting vials and pre-filled syringes were stored under different conditions. The vials were stored for 12 days at 5, 25 and 40° C. Pre-filled syringes were stored at 5° C. only. After 12 days of storage, all different concepts (both vials and syringes) were evaluated for resuspendability (time to reach visually homogenous suspension). The results are shown in Table 7.

TABLE 7
Resuspendability after 12 days of storage at different conditions

| Surfactant/ | Resuspendability (seconds) | | | |
|---|---|---|---|---|
| wetting agent | 5° C. | RT | 40° C. | SC syringes (5° C.) |
| Polysorbate 20 | >5 < 10 | >5 < 10 | <5 | >15 < 20 |
| Poloxamer 338 | >5 < 10 | >15 < 20 | >5 < 10 | >15 < 20 |
| Vitamin E | >5 < 10 | >5 < 10 | <5 | >10 < 15 |
| TPGS | | | | |

### Example 6: Preparation of Formulation Examples—Drug Substance Concentration

One individual vial (volume=50 mL) was prepared. In this vial, 3.1350 g (200 mg/mL eq.) of calcium; {4-[(5,6-diphenylpyrazin-2-yl)(propan-2-yl)amino]butoxy}acetate was weighed. Next to the active ingredient, 45 g of 1 mm Zirconium beads was added to the vial. In a next step 12 mL of a 125% over-concentrated stock solution of Polysorbate 20, PEG 4000 and Mcilvaine buffer was added to the vial. The composition of the stock solution is shown in Table 8 below.

TABLE 8
Composition of the tested 125% over-concentrated Polysorbate
20/PEG 4000/McIlvaine buffer (pH 8) stock solution

| | Concentration (mg/mL) |
|---|---|
| | Experimental ID |
| Ingredient | n001-00134 |
| Polysorbate 20 | 12.50 |
| PEG 4000 | 93.75 |
| Disodium hydrogen | 10.00 |
| phosphate anhydrous | |
| Citric acid monohydrate | 0.2125 |
| 1N HCl | q.s. ad pH 8 |
| Purified water | q.s. |

In a last step, 3 mL of purified water was added to the vial. The vial was put on a roller mill with a rotational speed of 300 rpm. To reach a target particle size (i.e. dv50) of 10 μm, 6 minutes of milling was needed. An overview of the resulting particle size (obtained with Mastersizer® 3000) is shown in Table 9.

TABLE 9
Overview of the different milling
times and the resulting particle sizes.

| Experimental | Milling | Particle size | | |
|---|---|---|---|---|
| ID | time | (Dv10, μm) | (Dv50, μm) | (Dv90, μm) |
| n001-001 | 6 min. | 3.6 | 10.3 | 34.7 |

The milled suspension was harvested into a 8 mL vial and final particle size was determined with Mastersizer® 3000. The resulting particle size distribution is shown in FIG. 6.

### Example 7: Preparation of Formulation Examples—Resuspending Agent

3 separate vials (volume=50 mL) were prepared with varying concentrations of PEG 4000 (50, 75 and 100 mg/mL). In each vial, 1.568 g (100 mg/mL eq.) of calcium; {4-[(5,6-diphenylpyrazin-2-yl)(propan-2-yl)amino]butoxy}acetate was weighed. Next to the active ingredient, 45 g of 1 mm Zirconium beads was added to each vial. In a next step 12 mL of a 125% over-concentrated stock solution of Polysorbate 20, PEG 4000 and TRIS buffer was added to each vial. The composition of the stock solution was dependent on the experiment. The composition of each stock solution is shown in Table 10 below.

TABLE 10
Composition of the tested 125% over-concentrated
Polysorbate 20/PEG 4000/TRIS buffer (pH 8) stock solutions

| | Concentration (mg/mL) | | |
|---|---|---|---|
| | Experimental ID | | |
| Ingredient | n001-00101 | n001-00119 | n001-00111 |
| Polysorbate 20 | 6.25 | | |
| PEG 4000 | 62.5 | 93.75 | 125 |

| | |
|---|---|
| Trometamol (TRIS) | 7.571 |
| 1N HCl | q.s. ad pH 8 |
| Purified water | q.s. |

In a last step, 3 mL of purified water was added to each vial. All 3 vials were put on a roller mill with a rotational speed of 300 rpm. To reach a target particle size (i.e. dv50) of 8 μm, different milling times were needed for each concept. An overview of the different milling times and the resulting particle sizes (obtained with Mastersizer® 3000) is shown in Table 11.

TABLE 11
Overview of the different milling
times and the resulting particle sizes.

| Experimental | Milling | Particle size | | |
|---|---|---|---|---|
| ID | time | (Dv10, μm) | (Dv50, μm) | (Dv90, μm) |
| n001-00101 | 30 min. | 2.7 | 7.5 | 24.0 |
| n001-00119 | 30 min. | 2.9 | 8.3 | 30.7 |
| n001-00111 | 26 min. | 2.84 | 8.02 | 29.8 |

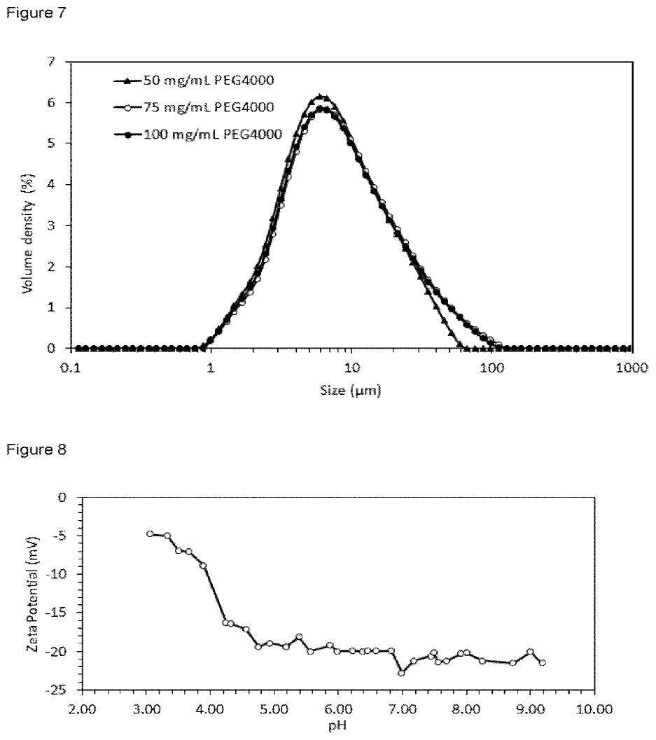

Each milled suspension was harvested in 5 mL vials and final particle size was determined with Mastersizer® 3000. An overlap of the resulting particle size distributions is shown in FIG. 7.

### Example 8: Characterization of Surface Charge by Measuring Zeta Potential

A suspension of calcium; {4-[(5,6-diphenylpyrazin-2-yl)(propan-2-yl)amino]butoxy}acetate (100 mg/mL) was prepared to a particle size Dv50 of around 1 micrometer. The suspension was diluted 10000 times in water. The pH was adjusted from pH 3.0 to pH 9.0 with either with HCl solution or NaOH solution. Zeta potential was measured using Zetasizer Ultra equipment from Malvern. FIG. 8 shows the results of zeta potential as function of pH of suspensions. The Zeta potential is an indicator for the stability of the suspension.

### Example 9: Lyophilisation of Suspension Concepts

To further improve the stability of the suspensions at room (or higher) temperature, feasibility of lyophilization of 4 suspension concepts were conducted. The suspension formulation components prior to lyophilization and the lyophilization program are shown in tables as below.

TABLE 12

| | Concept 1 | Concept 2 | Concept 3 | Concept 4 |
|---|---|---|---|---|
| Component | (mg/mL) | (mg/mL) | (mg/mL) | (mg/mL) |
| Selexipag metabolite | 100 | 100 | 100 | 100 |
| Ca-salt | | | | |
| Poloxamer 338 | — | 15 | — | 15 |
| Polysorbate 20 | 5 | — | 5 | — |
| PEG 4000 | 75 | 75 | 75 | 75 |
| Sodium hydrogen | 23.38 | 23.38 | 23.38 | 23.38 |
| phosphate | | | | |
| Citric acid monohydrate | 3.71 | 3.71 | 3.71 | 3.71 |
| Mannitol | — | — | 50 | 50 |
| Water for injection | q.s. ad | q.s. ad | q.s. ad | q.s. ad |
| | 1 mL | 1 mL | 1 mL | 1 mL |

TABLE 13
Lyophilization program

| | Tₛₜₐᵣₜ | T_{end} | Time | Ramping speed | Time | Vaccum SP | |
|---|---|---|---|---|---|---|---|
| Lyo step | (° C.) | (C) | (min) | (° C./min) | (hour) | (μbar) | Air supply |
| Pre-cooling of | Not required | | | | | | Sterile |
| shelves | | | | | | | compressed |
| Freezing | 20 | −40 | 200 | 0.3 | 3.33 | Atmospheric | air |
| Hold at −40° C. | −40 | −40 | 500 | / | 8.33 | Atmospheric | |
| Primary drying | −40 | −20 | 120 | 0.33 | 2 | 100 | |
| Hold at −20° C. | −20 | −20 | 2880 | / | 48 | 100 | |
| Secondary | −20 | 25 | 100 | 0.45 | 1.67 | 100 | |
| drying | | | | | | | |
| Hold at 25° C. | 25 | 25 | 300 | / | 5 | 100 | |
| Stoppering | | | | | | Atmospheric | |

The lyophilized concepts were easily reconstituted in water in 30 to 60 seconds by gentle shaking, and the particle size (Dv50) results were comparable before and after lyophilization. No particle aggregation was observed after lyophilization.

TABLE 14
Particle size distribution before and after lyophilization

| | Dv50 before | Dv50 after |
|---|---|---|
| Concept | lyo (μm) | lyo (μm) |
| 1 | 8.0 | 8.1 |
| 2 | 8.1 | 8.1 |
| 3 | 8.2 | 7.8 |
| 4 | 8.1 | 7.8 |

### Example 10: Sterilization of Drug Substance by Gamma-Irradiation

To evaluate if gamma-irradiation could be used to produce sterile product, different irradiation grades were tested on calcium; {4-[(5,6-diphenylpyrazin-2-yl)(propan-2-yl)amino]butoxy}acetate. In this study, calcium; {4-[(5,6-diphenylpyrazin-2-yl)(propan-2-yl)amino]butoxy}acetate (“COMPOUND”) was irradiated with three different grades (i.e. 5, 25 and 40 kGy). Each irradiation grade was applied on two different COMPOUND vials. One of the two vials for each grade was flushed with nitrogen for 10 seconds. One reference vial containing non-irradiated COMPOUND was included in the assay/purity analysis. All samples were analyzed immediately after irradiation (Table 15) and re-analyzed after 3 months of storage at room temperature.

TABLE 15
Initial Assay/purity results of gamma-irradiated
calcium; {4-[(5,6-diphenylpyrazin-2-yl)(propan-
2-yl)amino]butoxy}acetate (“COMPOUND”)

| | | Impurity, % | | |
|---|---|---|---|---|
| | Assay, % | RRT | RRT | RRT |
| Sample Name | COMPOUND | 1.16* | 1.32* | 1.34* |
| Reference-T0 | 95.3 | <0.05 | <0.05 | 0.09 |
| Reference-3 months | 94.4 | <0.05 | <0.05 | <0.05 |
| 5 kGy-T0 | 95.3 | 0.05 | <0.05 | 0.09 |
| 5 kGy-3 months | 94.0 | <0.05 | 0.06 | <0.05 |
| 5 kGy-T0 | 94.6 | 0.05 | <0.05 | 0.06 |
| (flushed with N2) | | | | |
| 5 kGy-3 months | 94.1 | <0.05 | 0.06 | <0.05 |
| (flushed with N2) | | | | |
| 25 kGy-T0 | 95.1 | 0.10 | 0.11 | 0.06 |
| 25 kGy-3 months | 94.2 | 0.07 | 0.12 | 0.06 |
| 25 kGy-T0 | 95.6 | <0.05 | 0.13 | 0.09 |
| (flushed with N2) | | | | |
| 25 kGy-3 months | 93.6 | <0.05 | 0.05 | 0.08 |
| (flushed with N2) | | | | |
| 40 kGy-T0 | 94.3 | 0.13 | 0.14 | 0.06 |
| 40 kGy-3 months | 94.3 | 0.08 | 0.15 | 0.10 |
| 40 kGy-T0 | 94.5 | 0.12 | 0.15 | 0.07 |
| (flushed with N2) | | | | |
| 40 kGy-3 months | 92.9 | 0.08 | 0.14 | 0.10 |
| (flushed with N2) | | | | |

RRT 1.16* is an impurity with a relative retention time of 1.16 min (1-Butanol, 4-[(5,6-diphenyl-2-pyrazinyl)(1-methylethyl)amino]-)
RRT 1.35* is an impurity with a relative retention time of 1.31 min
RRT 1.34* is an impurity with a relative retention time of 1.34 min (acetic acid, 2-[4-[(5,6-diphenyl-2-pyrazinyl)(1-methylethyl)amino]butoxy]-, ethyl ester)

A second study was conducted on calcium: {4-[(5,6-diphenylpyrazin-2-yl)(propan-2-yl)amino]butoxy}acetate where three different irradiation grades were applied (i.e 5, 25 and 40 kGy). Results are shown in Table 16.

TABLE 16
Initial Assay/purity results of gamma-irradiated
calcium; {4-[(5,6-diphenylpyrazin-2-yl)(propan-
2-yl)amino]butoxy}acetate (“COMPOUND”)

| Irradiation | | Assay, % | Impurity, % | | |
|---|---|---|---|---|---|
| grade | | COMPOUND | RRT 1.15* | RRT 1.16* | RRT 1.32* |
| 5 | kGy | 104.4 | <0.05 | <0.05 | <0.05 |
| 25 | kGy | 104.3 | 0.06 | 0.09 | 0.07 |
| 40 | kGy | 103.7 | 0.06 | 0.12 | 0.10 |

RRT 1.15* is an impurity with a relative retention time of 1.15 min (N-isopropyl-5,6-diphenyl-pyrazin-2-amine)
RRT 1.16* is an impurity with a relative retention time of 1.16 min (1-Butanol, 4-[(5,6-diphenyl-2-pyrazinyl)(1-methylethyl)amino]-)
RRT 1.32* is an impurity with a relative retention time of 1.32 min (N-isopropyl-N-(4-methoxybutyl)-5,6-diphenyl-pyrazin-2-amine)

Based on the results in Table 15 and 16, it can be concluded that with increasing irradiation grade, three impurities/degradation products gradually increase. Nevertheless, the chemical stability of calcium; {4-[(5,6-diphenylpyrazin-2-yl)(propan-2-yl)amino]butoxy}acetate is considered acceptable, demonstrating the feasibility of sterilization of drug substance using gamma-irradiation.

Analytical Method used: The assay and the impurities of calcium salt of selexipag metabolite before (ref) and after gamma irradiation were analysed using reversed phase chromatography. Sample preparation was done by weighing 50 mg of sample in a 500 mL flask. Approximately 100 mL pH7 phosphate buffer:ACN (50:50) was added whereafter the flask was shaken mechanically for at least 30 minutes until complete dissolution. Dilute to volume with pH7 phosphate buffer:ACN (50:50). The resulting solution was diluted 5× by pipetting 5 mL into a 25 mL flask and diluting with the same dilution solvent. The reference solution is made following the same sample preparation, but using calcium salt of selexipag metabolite (“API”).

Analysis was performed using a Waters UPLC H-Class equipped with DAD detector, column manager and auto-sampler. Separation was done on a Acquity UPLC BEH C18 (2.1×150 mm, 1.7 μm) analytical column using a column temperature of 60° C. DAD detector was set on 230 nm. A 10 mM NH₄Ac:ACN:MeOH (950:38:12) solution was used as mobile phase A. A 10 mM NH₄Ac:ACN:MeOH (50:710:240) solution was used as mobile phase B. A linear gradient program of 26 minutes was applied starting in which the mobile phase B increases from 5% to 100% in 20 minutes. Hereafter the concentration of mobile phase B was brought back to 5% in 1 minute followed by an equilibration time of 5 minutes. The applied flow rate is 0.30 mL/min. An injection volume of 7 μL was used for the analysis. The assay value of the sample is calculated according following formula:

%=[API peak responseₛₐₘₚₗₑ×conc_{ref}×purity_{ref}×100%]/[peak response_{ref}×concₛₐₘₚₗₑ]

The concentration of an impurity is calculated according following formula:

%=[Impurity peak responseₛₐₘₚₗₑ×conc_{ref}×purity_{ref}×100%]/[peak response_{ref}×concₛₐₘₚₗₑ]

### Example 11: Sterilization of Drug Product by Gamma-Irradiation

Although feasibility for gamma-irradiation of drug substance is demonstrated, terminal sterilization is still preferred. For this reason, both gamma-irradiation and autoclavation (steam sterilization) of the final drug product need to be investigated. To evaluate the chemical stability of the final drug product during gamma-irradiation, 2 different concepts with calcium; {4-[(5,6-diphenylpyrazin-2-yl)(propan-2-yl)amino]butoxy}acetate having a particle size Dv50 of 8 micrometer were subjected to an irradiation grade of 40 kGy and 60 kGy. An overview of the study with the associating assay/purity results is shown in Table 17.

TABLE 17
Overview of drug product (i.e. formulation) concepts subjected to gamma-irradiation with the corresponding assay/purity results.

| Formulation | | | | | | Impurity, % | | | | | |
|---|---|---|---|---|---|---|---|---|---|---|---|
| Irradiation | | Resuspending | | | Assay, % | RRT | RRT | RRT | RRT | RRT | RRT |
| grade | API | agent | Surfactant | Buffer | COMPOUND. | 0.60 | 0.72 | 1.11* | 1.15 | 1.16 | 1.32 |
| Ref. | 100 mg/mL | 75 mg/mL | 5 mg/mL | Phosphate- | 105.4 | <0.05 | <0.05 | <0.05 | <0.05 | <0.05 | <0.05 |
| 40 kGy | eq. | PEG | Polysorbate | citrate | 101.9 | <0.05 | <0.05 | 0.45 | <0.05 | 0.06 | 0.12 |
| 60 kGy | Selexipag | 4000 | 20 | (pH 8) | 102.0 | <0.05 | <0.05 | 0.70 | <0.05 | <0.05 | 0.19 |
| Ref. | metabolite | | 15 mg/mL | | 101.0 | <0.05 | <0.05 | <0.05 | <0.05 | <0.05 | <0.05 |
| 40 kGy | Ca-salt | | Poloxamer 338 | | 97.4 | <0.05 | <0.05 | 0.39 | <0.05 | 0.05 | 0.12 |
| 60 kGy | | | | | 98.3 | 0.05 | 0.05 | 0.65 | <0.05 | <0.05 | 0.18 |

RRT 0.60* is an impurity with a relative retention time of 0.60 min
RRT 0.72* is an impurity with a relative retention time of 0.72 min
RRT 1.11* is an impurity with a relative retention time of 1.11 min
RRT 1.15* is an impurity with a relative retention time of 1.15 min (N-isopropyl-5,6-diphenyl-pyrazin-2-amine)
RRT 1.16* is an impurity with a relative retention time of 1.16 min (1-Butanol, 4-[(5,6-diphenyl-2-pyrazinyl)(1-methylethyl)amino]-)
RRT 1.32* is an impurity with a relative retention time of 1.32 min (N-isopropyl-N-(4-methoxybutyl)-5,6-diphenyl-pyrazin-2-amine)

Several impurities are formed when the different concepts are subjected to gamma-irradiation. In all gamma-irradiated concepts. RRT 1.11 can be found. Based on identification results, this impurity is an esterification product of PEG 4000 and calcium; {4-[(5,6-diphenylpyrazin-2-yl)(propan-2-yl)amino]butoxy}acetate (i.e. PEG adduct).

Impurities RRT 1.15 and 1.16 are less present during gamma-irradiation of the formulated drug product, compared with the unformulated drug substance. The assumption is that the radicals present in the drug product have a higher tendency to react with PEG 4000, hence less formation of RRT 1.15. Furthermore, there is an assumption that RRT 1.16 will convert to RRT 1.32 in the drug product.

Based on the results in Table 17, it can be concluded that with increasing irradiation grade, the impurities/degradation products gradually increase. Nevertheless, the chemical stability of calcium; {4-[(5,6-diphenylpyrazin-2-yl)(propan-2-yl)amino]butoxy}acetate in the drug product are considered acceptable, demonstrating the feasibility of sterilization of drug product using gamma-irradiation.

Analytical Method used: The assay and the impurities of the gamma irradiated and non-gamma irradiated (ref) calcium salt of selexipag metabolite suspension (formulation) were analysed using reversed phase chromatography. Sample preparation was done by weighing 0.5 mL suspension in a 500 mL flask. Approximately 200 mL pH7 phosphate buffer:ACN (50:50) was added whereafter the flask was shaken mechanically for at least 30 minutes until complete dissolution. Dilute to volume with pH7 phosphate buffer:ACN (50:50). The resulting solution was diluted 5× by pipetting 5 mL into a 25 mL flask and diluting with the same dilution solvent. The reference solution is made following the same sample preparation, but using calcium salt of Selexipag Metabolite (see example 10).

Analysis was performed using a Waters UPLC H-Class equipped with DAD detector, column manager and auto-sampler. Separation was done on a Acquity UPLC BEH C18 (2.1×150 mm, 1.7 μm) analytical column using a column temperature of 60° C. DAD detector was set on 230 nm. A 10 mM NH₄Ac:ACN:MeOH (950:38:12) solution was used as mobile phase A. a 10 mM NH₄Ac:ACN:MeOH (50:710:240) solution was used as mobile phase B. A linear gradient program of 26 minutes was applied starting in which the mobile phase B increases from 5% to 100% in 20 minutes. Hereafter the concentration of mobile phase B was brought back to 5% in 1 minute followed by an equilibration time of 5 minutes. The applied flow rate is 0.30 mL/min. An injection volume of 7 μL was used for the analysis. The assay value of the sample is calculated according following formula:

%=[API peak responseₛₐₘₚₗₑ×conc_{ref}×purity_{ref}×100%]/[peak response_{ref}×concₛₐₘₚₗₑ×dose claim]

The concentration of an impurity is calculated according following formula:

%=[impurity peak responseₛₐₘₚₗₑ×conc_{ref}×purity_{ref}×100%]/[peak response_{ref}×concₛₐₘₚₗₑ×dose claim]

### Example 12: Sterilization of Drug Product by Autoclavation (Steam Sterilization)

In parallel with gamma-irradiation of the final drug product (formulated calcium; {4-[(5,6-diphenylpyrazin-2-yl)(propan-2-yl)amino]butoxy}acetate having a particle size Dv50 of 8 micrometer), autoclavation (steam sterilization) was evaluated as well. Autoclavability (122° C., 15 min.) was investigated on 2 different concepts in two separate studies, as shown in Table 18.

TABLE 18
Overview of drug product concepts subjected to autoclavation (steam
sterilization) with the corresponding assay/purity results.

| Formulation | | | | | | Impurity, % | | | | | |
|---|---|---|---|---|---|---|---|---|---|---|---|
| | | Resuspending | | | Assay, % | RRT | RRT | RRT | RRT | RRT | RRT |
| Concept | API | agent | Surfactant | Buffer | COMPOUND | 0.60 | 0.72 | 1.11* | 1.15 | 1.16 | 1.32 |
| 135-1 | 100 mg/mL | 75 mg/mL | 5 mg/mL | Phosphate/ | 97.5 | <0.05 | <0.05 | <0.05 | <0.05 | <0.05 | <0.05 |
| | eq. | PEG | Polysorbate 20 | citrate | | | | | | | |
| 135-2 | Selexipag | 4000 | 15 mg/mL | pH 8 | 96.1 | <0.05 | <0.05 | <0.05 | <0.05 | <0.05 | <0.05 |
| | Ca-salt | | Poloxamer 338 | | | | | | | | |
| 142-5 | | | 5 mg/mL | | 103.3 | <0.05 | <0.05 | <0.05 | <0.05 | <0.05 | <0.05 |
| | | | Polysorbate 20 | | | | | | | | |
| 142-2 | | | 15 mg/mL | | 97.4 | <0.05 | <0.05 | <0.05 | <0.05 | <0.05 | <0.05 |
| | | | Poloxamer 338 | | | | | | | | |

RRT 0.60* is an impurity with a relative retention time of 0.60 min
RRT 0.72* is an impurity with a relative retention time of 0.72 min
RRT 1.11* is an impurity with a relative retention time of 1.11 min
RRT 1.15* is an impurity with a relative retention time of 1.15 min (N-isopropyl-5,6-diphenyl-pyrazin-2-amine)
RRT 1.16* is an impurity with a relative retention time of 1.16 min (1-Butanol, 4-[(5,6-diphenyl-2-pyrazinyl)(1-methylethyl)amino]-)
RRT 1.32* is an impurity with a relative retention time of 1.32 min (N-isopropyl-N-(4-methoxybutyl)-5,6-diphenyl-pyrazin-2-amine)

Results show that no impurities are formed during autoclavation and hence autoclavation (steam sterilization) does not impact chemical stability of the different studied drug concepts. Not only the effect upon chemical stability, but upon resuspendability was evaluated as well for the process of autoclavation (steam sterilization). The tested samples of experiment 142 were evaluated for resuspendability right after autoclavation (T0) and after 14 days of storage at 5° C. An overview of the results is shown in Table 19. Time needed for resuspendability was acceptable at both timepoints.

TABLE 19
Resuspendability results of drug product concepts
after subjected to autoclavation (steam sterilization)

| | Sampling | Resuspendability |
|---|---|---|
| Concept | time | (Seconds) |
| 142-5 | T0 | 15-20 |
| | 14 days | 15-20 |
| | (storage | |
| | at 5° C.) | |
| 142-2 | T0 | 15-20 |
| | 14 days | 15-20 |
| | (storage | |
| | at 5° C.) | |

Based on the results in Table 18 and 19, it can be concluded that calcium; {4-[(5,6-diphenylpyrazin-2-yl)(propan-2-yl)amino]butoxy}acetate is chemically stable in the drug product after the sterilization of drug product using autoclavation (steam sterilization). No aggregation was formed and the drug product can be easily resuspended after autoclavation.

### Example 13: Conclusion

To guarantee sterility of the final drug product (formulated calcium; {4-[(5,6-diphenylpyrazin-2-yl)(propan-2-yl)amino]butoxy}acetate having a particle size Dv50 of 8 micrometer), feasibility of three potential pathways was studied: gamma-irradiation of drug product and heat sterilization of drug product. To demonstrate feasibility, both physical (i.e. resuspendability and particle size) and chemical stability (i.e. formation of impurities) should be guaranteed, and the results are shown in Table 20.

TABLE 20
Summary of assay/impurity results for drug substance (not formulated) and
drug product (formulated) sterilized by gamma-irradiation and autoclavation

| | | | Impurity, % | | | | | |
|---|---|---|---|---|---|---|---|---|
| | | Assay, % | RRT | RRT | RRT | RRT | RRT | RRT |
| Sample Name | Concept | COMPOUND | 0.60 | 0.72 | 1.11 | 1.15 | 1.16 | 1.32 |
| DP - PX338 | 142-1 | 101 | <0.05 | <0.05 | <0.05 | <0.05 | <0.05 | <0.05 |
| (reference) | | | | | | | | |
| DP - PX338 | 142-2 | 97.4 | <0.05 | <0.05 | <0.05 | <0.05 | <0.05 | <0.05 |
| (autoclavation) | | | | | | | | |
| DP - PX338 | 142-10 | 97.4 | <0.05 | <0.05 | 0.39 | <0.05 | 0.05 | 0.12 |
| (γ-irradiation/ | | | | | | | | |
| 40 kGy) | | | | | | | | |
| DP - PX338 | 142-3 | 98.3 | 0.05 | 0.05 | 0.65 | <0.05 | <0.05 | 0.18 |
| (γ -irradiation/ | | | | | | | | |
| 60 kGy) | | | | | | | | |
| DP - PS 20 | 142-4 | 105.4 | <0.05 | <0.05 | <0.05 | <0.05 | <0.05 | <0.05 |
| (reference) | | | | | | | | |
| DP - PS 20 | 142-5 | 103.3 | <0.05 | <0.05 | <0.05 | <0.05 | <0.05 | <0.05 |
| (autoclavation) | | | | | | | | |
| DP - PS 20 | 142-12 | 101.9 | <0.05 | <0.05 | 0.45 | <0.05 | 0.06 | 0.12 |
| (γ-irradiation/ | | | | | | | | |
| 40 kGy) | | | | | | | | |
| DP - PS 20 | 142-6 | 102 | <0.05 | <0.05 | 0.7 | <0.05 | <0.05 | 0.19 |
| (γ-irradiation/ | | | | | | | | |
| 60 kGy) | | | | | | | | |

DP: drug product (formulated)
DS: drug substance (not formulated)
PX338: Poloxamer 338
PS20: polysobate 20
RRT 0.60* is an impurity with a relative retention time of 0.60 min
RRT 0.72* is an impurity with a relative retention time of 0.72 min
RRT 1.11* is an impurity with a relative retention time of 1.11 min
RRT 1.15* is an impurity with a relative retention time of 1.15 min (N-isopropyl-5,6-diphenyl-pyrazin-2-amine)
RRT 1.16* is an impurity with a relative retention time of 1.16 min (1-Butanol, 4-[(5,6-diphenyl-2-pyrazinyl)(1-methylethyl)amino]-)
RRT 1.32* is an impurity with a relative retention time of 1.32 min (N-isopropyl-N-(4-methoxybutyl)-5,6-diphenyl-pyrazin-2-amine)

Based on Table 20, calcium; {4-[(5,6-diphenylpyrazin-2-yl)(propan-2-yl)amino]butoxy}acetate is chemically stabile by autoclavation (steam sterilization) of the final drug product. With gamma-irradiation of the drug product, impurity levels are relatively high but still in an acceptable range. Therefore, it can be concluded that the final drug product can be sterilized with both autoclavation (steam sterilization) and gamma irradiation.

1. A pharmaceutical composition in the form of an aqueous suspension comprising
(a) calcium; {4-[(5,6-diphenylpyrazin-2-yl)(propan-2-yl)amino]butoxy}acetate of formula (I), or a pharmaceutically acceptable hydrate or solvate thereof: 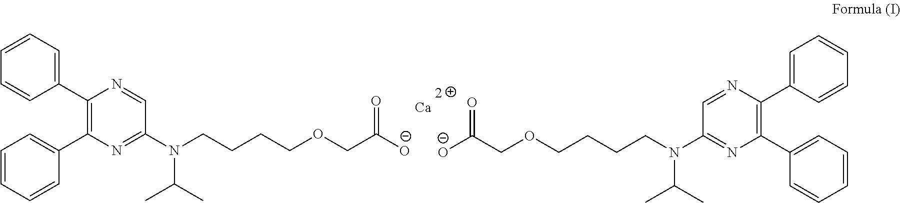
having a particle size distribution Dv50 of 1 to 40 μm;
(b) a surfactant and/or wetting agent; and
(c) a pharmaceutically acceptable aqueous carrier,
wherein the pharmaceutical composition has a pH in the range of 6 to 8.5.

2. The pharmaceutical composition of claim 1, further comprising a resuspending agent.

3. The pharmaceutical composition of claim 1, wherein the particle size distribution Dv50 is from 5 to 15 μm.

4. The pharmaceutical composition of claim 1, wherein the surfactant and/or wetting agent is selected from the group consisting of poloxamer 338, polysorbate 20, and Vitamin E TPGS, or a mixture thereof.

5. The pharmaceutical composition of claim 2, wherein the resuspending agent is selected from the group consisting of polyethylene glycol (PEG), carmellose sodium, and poloxamer, or a mixture thereof.

6. The pharmaceutical composition of claim 2, wherein the resuspending agent is selected from the group consisting of PEG 4000, PEG 3350, PEG 6000, PEG 8000, PEG 20000, and carmellose sodium, or a mixture thereof.

7. The pharmaceutical composition of claim 6, wherein the resuspending agent comprises PEG 4000.

8. The pharmaceutical composition of claim 1, wherein the aqueous carrier comprises a buffering and/or pH adjusting agent.

9. The pharmaceutical composition of claim 8, wherein the buffering and/or pH adjusting agent is selected from the group consisting of disodium hydrogen phosphate, citric acid, tris(hydroxymethyl)aminomethane, HCl, and NaOH, or a mixture thereof.

10. The pharmaceutical composition of claim 8, wherein the buffering and/or pH adjusting agent comprises tris(hydroxymethyl)aminomethane.

11. The pharmaceutical composition of claim 8, wherein the buffering agent is at a buffer strength of 5 to 100 millimolar (mM).

12. The pharmaceutical composition of claim 1, further comprising an oxygen scavenger.

13. The pharmaceutical composition of claim 12, wherein the oxygen scavenger comprises methionine.

14. The pharmaceutical composition of claim 1, further comprising an isotonizing agent.

15. The pharmaceutical composition of claim 14, wherein the isotonizing agent comprises an inorganic salt.

16. The pharmaceutical composition of claim 2, wherein the surfactant and/or wetting agent comprises a polysorbate; the buffering and/or pH adjusting agent comprises tris(hydroxymethyl)aminomethane; the resuspending agent comprises a polyethylene glycol, and, optionally, the pharmaceutical composition further comprises an oxygen scavenger comprising methionine, and an isotonizing agent comprising an inorganic salt.

17. The pharmaceutical composition of claim 1, comprising:
(a) from 2% to 50% (w/v), or from 2% to 30% (w/v), or from 2% to 15% (w/v) or from 2.5% to 10% (w/v) of calcium; {4-[(5,6-diphenylpyrazin-2-yl)(propan-2-yl)amino]butoxy}acetate or a pharmaceutically acceptable hydrate or solvate thereof; wherein the w/v is calculated on the basis of its anhydrous form;
(b) from 0.5% to 20% (w/v), or from 0.5% to 15% (w/v), or from 0.5% to 12% (w/v), or 0.5% to 10%, or from 0.5% to 8% (w/v), or from 0.5% to 7% (w/v), or from 0.5% to 6% (w/v), or from 0.5% to 5% (w/v), or from 0.5% to 4% (w/v), or from 0.5% to 3% (w/v) of a surfactant and/or wetting agent, or a mixture of surfactants and/or wetting agents;
(c) from 0% to 30% (w/v), or from 1% to 30% (w/v), or from 1% to 20% (w/v), or from 1 to 15% (w/v), or from 3 to 10% (w/v) of a resuspending agent or a mixture of resuspending agents;
(d) from 0 to 100 mM, or from 5 to 50 mM, or from 10 to 50 mM of a buffering agent, or mixtures thereof; and
(e) water for injection q.s. ad 100%.

18. A process for preparing a pharmaceutical composition of claim 1, said process comprising the steps of:
(a) adding a crystalline form of calcium; {4-[(5,6-diphenylpyrazin-2-yl)(propan-2-yl)amino]butoxy}acetate, or a pharmaceutically acceptable hydrate or solvate thereof, to a liquid medium comprising a surfactant and/or wetting agent, optionally a resuspending agent; and a pharmaceutically acceptable aqueous carrier at a pH in the range of 6 to 8.5, to form a premix; and
(b) subjecting the premix to mechanical means in the presence of a grinding medium to reduce the average effective particle size.

19. A process for preparing a lyophilized pharmaceutical composition, said process comprising the steps of freezing the pharmaceutical composition of claim 1, followed by a drying step comprising applying a vacuum.

20. A lyophilized pharmaceutical composition obtainable by the process of claim 19.

21. A method of treating pulmonary hypertension in a patient in need thereof, comprising administering a therapeutically effective amount of a pharmaceutical composition to the patient, wherein the pharmaceutical composition is in the form of an aqueous suspension comprising,
calcium; {4-[(5,6-diphenylpyrazin-2-yl)(propan-2-yl)amino]butoxy}acetate of formula (I), or a pharmaceutically acceptable hydrate or solvate thereof:

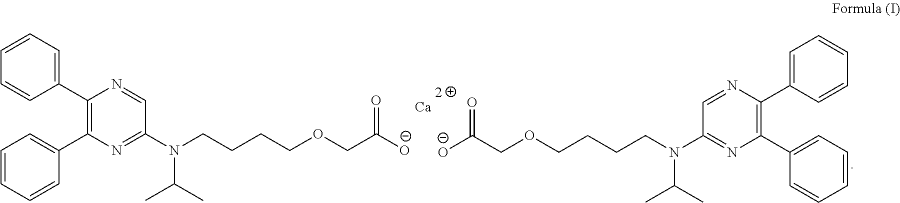

22. The method of claim 21, wherein the pulmonary hypertension is pulmonary arterial hypertension (PAH).

23. The method of claim 21, wherein the pulmonary hypertension is chronic thromboembolic pulmonary hypertension (CTEPH).

24. The method of claim 21, wherein the pharmaceutical composition is an intramuscular or subcutaneous injectable.

25. The method of claim 24, wherein the pharmaceutical composition is administered at a time interval between each administration of one week to three months.

26. The method of claim 25, wherein the time interval is two weeks to one month.

27. The method of claim 21, wherein the pharmaceutical composition comprises microparticles of calcium; {4-[(5,6-diphenylpyrazin-2-yl)(propan-2-yl)amino]butoxy}acetate of formula (I), or a pharmaceutically acceptable hydrate or solvate thereof, having a particle size distribution Dv50 of 1 to 40 μm.

28. The method of claim 27, wherein the particle size distribution Dv50 is from 5 to 15 μm.

29. The method of claim 27, wherein the microparticles are suspended in an aqueous medium comprising a surfactant and/or wetting agent; a buffering and/or pH adjusting agent; and optionally, a resuspending agent, an oxygen scavenger, or an isotonizing agent, wherein the pharmaceutical composition has a pH in the range of 6 to 8.5.

30. The method of claim 29, wherein the surfactant and/or wetting agent comprises a polysorbate, the resuspending agent comprises a polyethylene glycol, the buffering and/or pH adjusting agent comprises tris(hydroxymethyl)aminomethane, the oxygen scavenger comprises methionine, and the isotonizing agent comprises an inorganic salt.
